(19) United States
(12) Patent Application Publication
(10) Pub. No.: US 2024/0301354 A1
(43) Pub. Date: Sep. 12, 2024

(54) SYSTEM AND METHOD FOR CONVERTING ADIPOSE DERIVED MESENCHYMAL STEMS CELLS TO HEMATOPOIETIC STEM/PROGENITOR CELLS AND DIFFERENTIATING INTO BLOOD CELLS AND APPLICATIONS OF SAME

(71) Applicant: UL Med Inc., Norcross, GA (US)

(72) Inventors: WEI LI, Norcross, GA (US); Shaojin You, Norcross, GA (US); Michael Chengnan Lin, Norcross, GA (US); Annie You, Norcross, GA (US)

(21) Appl. No.: 18/601,387

(22) Filed: Mar. 11, 2024

Related U.S. Application Data
(60) Provisional application No. 63/451,269, filed on Mar. 10, 2023.

(51) Int. Cl. C12N 5/0789; C12M 1/33; C12N 5/078; C12N 13/00

(52) U.S. Cl. CPC C12N 5/0647; C12M 45/02; C12N 5/0641; C12N 13/00; C12N 2500/84; C12N 2501/105; C12N 2501/22; C12N 2501/2303; C12N 2501/26; C12N 2501/33; C12N 2501/999; C12N 2506/1384

(57) ABSTRACT

A method of producing hematopoietic stem/progenitor cells (HS/PCs) and blood cells from adipose derived mesenchymal stem cells (Ad MSCs). The method comprises obtaining biological cells of a subject; expanding the biological cells; obtaining a pure Ad MSCs cell line from the expanded biological cells; converting the pure Ad MSCs cell line into HS/PCs in vitro; expanding the converted HS/PCs in vitro; and obtaining the expanded HS/PCs.

### CROSS-REFERENCE TO RELATED PATENT APPLICATIONS

This application claims priority to and the benefit of U.S. Provisional Patent Application Ser. No. 63/451,269, filed Mar. 10, 2023, which is incorporated herein in its entirety by reference.

### FIELD OF THE INVENTION

The present disclosure relates generally to the field of biotechnology, and more particularly to a system and/or a method for converting adipose derived mesenchymal stem cells (Ad MSCs) to hematopoietic stem/progenitor cells (HS/PC) and differentiating into blood cells.

### BACKGROUND OF THE INVENTION

The background description provided herein is for the purpose of generally presenting the context of the present invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

Recent advancements in stem cell technology using human pluripotent stem cells (hPSCs) and multipotent mesenchymal stem cells (MSCs) open a new door for patients suffering from diseases and disorders that have yet to be treated. These cells were first discovered by Arnold L. Caplan et al in mouse bone marrow (BM). Protocols were subsequently established to directly culture this subpopulation of stromal cells from BM in vitro. Since then, MSCs have been found in and derived from different human tissue sources, including adipose tissue (AT), umbilical cord (UC), UC blood, placenta, dental pulp, and amniotic fluid, etc. Among them, MSCs derived from AT, BM, and UC are the most studied MSCs in human clinical trials. The percentage of MSCs in BM mononuclear cells ranges from 0.001 to 0.01% following gradient centrifugation. The number of MSCs in AT is at least 500 times higher than that in BM, with approximately 5,000 MSCs per 1 g of AT. To date, 1426 registered clinical trials spanning different trial phases have used MSCs for therapeutic purposes. As supported by a large body of preclinical studies and advancements in conducting clinical trials, MSCs have been proven to be effective in the treatment of numerous diseases, including nervous system and brain disorders, pulmonary diseases, cardiovascular conditions, wound healing, etc.

Human AT was first viewed as a passive reservoir for energy storage and later as a major site for sex hormone metabolism, the production of endocrine factors (such as adipsin and leptin), and a secretion source of bioactive peptides known as adipokines. It is now clear that AT functions as a complex and highly active metabolic and endocrine organ, orchestrating numerous different biological features. In addition to adipocytes, AT contains hematopoietic-derived progenitor cells, connective tissue, nerve tissue, stromal cells, endothelial cells, MSCs, and pericytes. MSCs are key elements in the bone marrow niche where they interact with hematopoietic stem progenitor cells (HSPCs) by offering physical support and secreting soluble factors, which control HSPC maintenance and fate. In light of HSPC supportive properties, MSCs have been employed in phase I/II clinical trials of hematopoietic stem cell transplantation (HSCT) to facilitate engraftment of hematopoietic stem cells (HSCs). Moreover, they have been utilized to co-culture and expand ex vivo HSCs before clinical use. More recently, several studies have demonstrated that MSCs display broad and potent immunoregulatory properties both in vitro and in vivo, which have prompted their clinical uses in the setting of hematopoietic stem cell transplantation (HSCT), especially for the treatment of immune-mediated complications.

Adipose tissue is composed of lipid-filled mature adipocytes and other nonadipocyte cells, called the stromal vascular fraction (SVF). The SVF is not a fully defined cell population consisting of various types of cells, including stem cells, which have multiple differentiation potentials making these cells (MSCs) attractive for tissue engineering and cell therapies. Under the influence of appropriate growth factors and microenvironmental conditions, they relatively easily differentiate into chondrocytes and osteocytes. The differentiation potential of these cells, however, depends on many factors, including the age and sex of the donor, and the anatomical location of the fat. AD-MSCs isolated from mice were cultured as feeders with Lin-Sca-1⁺c-kit⁺ (HSPC) cells from mice for 2 and 5 weeks. In vitro study showed that AD-MSCs had high-Jagged-1 expression and promoted LSK cell proliferation; and in vivo study, AD-MSCs facilitated hematopoietic recovery and promoted the survival of NOD/SCID mice. According to previous reports, the estimated amount of HSPCs in total adipose tissues would be equivalent to approximately 0.2% of that of HSPCs in the total BM. Can Ad-MSCs be used practically as an alternative resource of HSPCs? So far, Ad-MSCs have not been successfully converted into functional HSPCs in large-scale for basic research and clinical uses in vitro.

The human-induced pluripotent stem cell (iPSC) technology was pioneered by Shinya Yamanaka and Kazutoshi Takahashi, who together showed in 2006 that the introduction of four specific genes (named Myc, Oct3/4, Sox2 and Klf4), encoding transcription factors could convert somatic cells into pluripotent stem cells. However, there are still challenges in reprogramming cells to iPSC, such as low efficiency, genomic insertion, tumorigenicity. Subsequent to this breakthrough, iPSC generation and culturing systems greatly improved with respect to delivery of the transgenes (footprint-free), reprogramming conditions (feeder-free, xeno-free), and the large-scale production and differentiation of iPSCs, leading toward clinical uses. Although iPSC-derived blood products are promising and a few iPSC-blood derivatives are entering clinical trials (e.g. T-cells/platelets), there are still various hurdles to overcome for most of the blood lineages. Progression is somewhat limited by the different protocols and procedures currently employed within different laboratories, making comparisons difficult. Cell and gene therapy has been developed significantly during the 21st century. Precision cell therapy based on autologous cells derived from a patient's own body holds great potential in personalized medicine. It is indeed expected to be a transformative period for this field. However, precision autologous cell therapy continues to face a significant challenge due to the lack of reliable sources, heterogeneity, scalability issues. In one aspect, significant difficulty exists in expanding hematopoietic stem cells (HSCs) in vitro, which are further compounded by immunogenicity and poor transgenic efficiency.

Currently, the most used hematopoietic stem cell therapy is autologous or allogeneic bone marrow transplantation. However, this treatment often fails to succeed due to the lack of autologous bone marrow conditions and/or matched allogeneic myeloid sources, as well as the side effects of allogeneic bone marrow transplantation, such as graft-versus-host disease (GVHD). Given the potentially serious complications of GVHD following the allogeneic stem cell transplantation, as well as widespread demand for autologous hematopoietic stem cells, finding other stem cell sources would be a true breakthrough and urgently needed for clinical practice.

Adipose-derived mesenchymal stem cells (Ad MSCs) share many similarities with mesenchymal stem cells derived from other sources like bone marrow but are harvested from adipose (fat) tissue. Ad MSCs possess a distinct transcriptome, and have demonstrated long-term self-renewal, serial clonogenicity, and multigerm layer differentiation potential. Indeed, in recent decades, the application of Ad MSCs in regenerative medicine is very active in the fields of bone, fat, liver, nerve and islet cell regeneration and repair. More than 131 clinical trials of Ad MSCs have been authorized by the FDA so far. Ad MSCs have rich resources (10-30% of body weight) and are easily accessible. For example, isolation of Ad MSCs typically involves collecting adipose tissue through liposuction, which is less invasive and often results in fewer complications compared to the collection of bone marrow.

Therefore, a heretofore unaddressed need exists in the art to address the aforementioned deficiencies and inadequacies, in particular, developing Ad MSCs as a new resource for deriving hematopoietic stem/progenitor cells (HS/PCs), available directly from mature adipose tissue which is body's largest resource. The mature adipose tissue not only bypass the need for embryos, but can be made in a patient-matched manner, which means that each individual could have their own pluripotent stem cell line. These unlimited supplies of autologous cells could be used to generate transplants without the risk of immune rejection. And thus bring a chance of survival to patients' uncurable disease.

### SUMMARY OF THE INVENTION

In light of the foregoing, this invention discloses a method of producing HS/PCs and differentiating blood cells from adipose derived mesenchymal stem cells (Ad MSCs). The method comprises obtaining biological cells of a subject; expanding the biological cells; obtaining a pure Ad MSCs cell line from the expanded biological cells; converting the pure Ad MSCs cell line into hematopoietic stem/progenitor cells (HS/PCs) in vitro; expanding the converted HS/PCs in vitro; and obtaining the expanded HS/PCs.

In one embodiment, the method further comprises differentiating the expanded HS/PCs into a blood cell line without co-culture with bone marrow derived HSCs; wherein the blood cell line comprises at least one of white blood cells (WBC), red blood cells (RBC), and platelets.

In one embodiment, the step of converting the pure Ad MSCs cell line into the HS/PCs does not use any exogenous genes.

In one embodiment, the step of converting the pure Ad MSCs cell line into the HS/PCs does not use any co-culturing with any other cells.

In one embodiment, the step of obtaining the pure Ad MSCs cell line comprises isolating the Ad MSCs single cell line from the biological sample; and culturing the Ad MSCs single cell line.

In one embodiment, the step of converting the pure Ad MSCs cell line into the HS/PCs comprises using a HS/PCs converting kit.

In one embodiment, the HS/PCs converting kit comprises a basic Ad MSCs culture medium; a basic nutrition supplement; and a basic converting supplement mixture.

In one embodiment, the step of converting the pure Ad MSCs cell line into the HS/PCs comprises preparing a HS/PC-C completely medium by mixing the basic nutrition supplement and the basic converting supplement mixture into the basic Ad MSCs culture medium; adding the HS/PC-C completely medium to the Ad MSCs single cell line obtained; and incubating the pure Ad MSCs cell line in the HS/PC-C completely medium for a period of time.

In one embodiment, the step of converting the pure Ad MSCs single cell line into the HS/PCs has a conversion efficiency of more than 10%.

In one embodiment, combined the converted HS/PCs with enhancing system has a higher gene transfection efficiency (about 25-fold higher than that of regular with converted HS/PCs or bone marrow derived).

In one embodiment, the converted HS/PCs comprises gene carry-on cells or gene delivery system.

In another aspect of the invention, a HS/PCs converting kit for converting adipose derived mesenchymal stem cells (Ad MSCs) into hematopoietic stem/progenitor cells (HS/PCs) comprises a basic Ad MSCs culture medium; a basic nutrition supplement; and a basic converting supplement mixture, wherein the kit converts the Ad MSCs into HS/PCs.

In one embodiment, the basic nutrition supplement comprises fetal bovine serum and horse serum.

In one embodiment, the basic converting supplement mixture comprises at least one of insulin, holo-transferrin, sodium selenite (ITS liquid), L-ascorbic acid, GM-CSF, SCF, VEGF, IGF-I, IGF-II, IL-3, Flt3-L, thrombopoietin (TPO), dexamethasone, fatty acid free BSA, I-thioglycerol, SB431542, CHIR99021, and Y-27632.

In one embodiment, the basic converting supplement mixture comprises insulin, holo-transferrin, sodium selenite (ITS liquid), L-ascorbic acid, GM-CSF, SCF, VEGF, IGF-I, IGF-II, IL-3, Flt3-L, thrombopoietin (TPO), dexamethasone, fatty acid free BSA, 1-thioglycerol, SB431542, CHIR99021, and Y-27632.

In yet another aspect of the invention, a red blood cells (RBCs) and platelets differentiation kit for differentiating hematopoietic stem/progenitor cells (HS/PCs) into RBCs and platelets comprises a basic cell culture medium; a basic nutrition supplement; and a RBCs supplement mixture; wherein the kit differentiates the HS/PCs into at least one of RBCs and platelets.

In one embodiment, the basic nutrition supplement comprises fetal bovine serum and horse serum.

In one embodiment, the RBCs supplement mixture comprises at least one of insulin, holo-transferrin, sodium selenite (ITS liquid), L-ascorbic acid, SCF, VEGF, IGF-I, IGF-II, IL-3, Flt3-L, thrombopoietin (TPO), dexamethasone, fatty acid free BSA, 1-thioglycerol, EPO, ferrous sulfate, PDGF BB, activin A.

In yet another embodiment of the invention, a white blood cells (WBCs) differentiation kit for differentiating hematopoietic stem/progenitor cells (HS/PCs) into WBCs, comprises a basic cell culture medium; a basic nutrition supplement; and a WBCs supplement mixture; wherein the kit differentiates the HS/PCs into WBCs.

In one embodiment, the basic nutrition supplement comprises fetal bovine serum and horse serum.

In one embodiment, the basic converting supplement mixture comprises at least one of insulin, holo-transferrin, sodium selenite (ITS liquid), L-ascorbic acid, SCF, VEGF, IGF-I, IGF-II, IL-3, Flt3-L, thrombopoietin (TPO), dexamethasone, fatty acid free BSA, 1-thioglycerol, BMP4, IL-7, IL-12, IL-11, IL-6, IL-2, FGF-b, TGF-a, TGF-b1, TGF-b2, TGF-b3, IL-1a, IL-1b, IL-4, IL-5, IL-8, IL-10, IL-12, IL-32a, mIL-36R2, GM-CSF.

In yet another aspect of the invention, an automatic multifunctional single cell processor and culture device comprises a housing; at least one small fluid volume dispenser system disposed inside the housing; and a tissue grinding system disposed below the small fluid volume dispenser system and in the housing; wherein the small fluid volume dispenser system is configured to dispense at least one fluid; and wherein the tissue grinding system receives the at least one fluid.

In one embodiment, the small fluid volume dispenser system is configured to dispense the at least one fluid in a volume between about 5-10 ml.

In one embodiment, the small fluid volume dispenser system comprises a cylinder houses one or more pre-filled cartridges.

In one embodiment, the small fluid volume dispenser system comprises a top needle disposed above the one or more pre-filled cartridges and a bottom needle disposed below the one or more pre-filled cartridges.

In one embodiment, the top needle and the bottom needle are configured to move in a vertical direction for piercing the pre-filled cartridges.

In one embodiment, the tissue grinding system comprises a vial mechanically connected to a motor.

In one embodiment, the vial houses a top grinder plate, a bottom grinder plate, a filter disposed below the top grinder plate and the bottom grinder plate.

In one embodiment, the top grinder plate and the bottom grinder plate are arranged to form a sample disposing place for receiving a biological sample.

In one embodiment, the tissue grinding system further comprises a tubing fluidly connected to a bottom of the vial on a first end.

In one embodiment, the tubing fluidly connected to a dispenser funnel on a second end.

In one embodiment, the device comprises a cellular flask rotator, wherein the cellular flask rotator comprises multiple magnetic nanoparticles.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 illustrates microscopic photos reflecting oil red O staining for human and mice Ad MSCs.

FIG. 2 shows Ad MSCs culture growth curve and Ad MSCs growth increased linearly after 7 days of culture.

FIG. 3 shows microscopic photographs reflecting that Ad MSCs growth increased linearly after 7 days of culture.

FIG. 4 shows markers positively expressed in Ad MSC from human breast fat tissue.

FIG. 5 shows markers positively expressed in Ad MSC from SS mice abdomen fat tissue.

FIG. 6 shows markers positively expressed in Ad MSC from CD45.1 mouse.

FIG. 7 shows converted HS/PCs using the UL™-h or m HS/PC-C completely medium for culture for human.

FIG. 8 shows converted HS/PCs using the UL™-h or m HS/PC-C completely medium for culture for mice.

FIG. 9 shows flow cytometry results for sorting Sca-1 positive cells from converted mouse HS/PCs.

FIG. 10 shows CD90.2 (blue) c-kit (CD117) (purple), CD48 (green), CD105 (red), CD150 (pink) markers are positively expressed in converted Sca-1 Positive mouse HS/PC(D).

FIG. 11 shows the FITC-Sca-1 marker (green) demonstrates positive expression in live cultured converted mouse HS/PCs(E).

FIG. 12 shows flow cytometry results reflecting that CD34 (green, red), CD90 (yellow, red) and CD49f (pink) markers are positive expressions in converted HS/PC from human cells.

FIG. 13 shows that Glycophorin A(CD235a) (green) marker was positively expressed in supernatant cells from human converted HS/PC under UL™-hRBC medium after 18 to 23 days culture.

FIG. 14 shows flow cytometry results demonstrating that converted human HS/PCs cultured in UL™-hRBC completely medium after 18 to 23 days.

FIG. 15 shows Glycophorin A (TER 119) (green) marker was positively expressed in supernatant cells from mouse converted HS/PC under UL™-mRBC Medium after 18 to 23 days culture.

FIG. 16 shows flow cytometry results demonstrating that converted mouse HS/PCs cultured in UL™-mRBC completely medium after 18 to 23 days.

FIG. 17 shows flow cytometry results demonstrating CD45.1 (red) and TER 119 (green) positive expression in CD45.1 mouse converted HS/PCs transplanted CD45.2 mouse blood cells on day 23.

FIG. 18 shows flow cytometry results demonstrating CD45.1 (red) and TER 119 (green) positive expression in CD45.1 mouse converted HS/PCs transplanted CD45.2 mouse blood cells on day 23.

FIG. 19 shows transplantation of the converted HS/PCs of CD45.1 mice into 6 of the CD45.2 mice.

FIG. 20 shows CBC results reflecting the cells in supernatant from mouse converted HS/PCs under UL™-mWBC medium at 25 days culture.

FIG. 21 reflects CBC results showing that the cells in supernatant from mouse converted HS/PCs under regular medium at day 25 days culture.

FIG. 22 shows flow cytometry analysis demonstrating that there is positive expression of the CD45.1 mice of leukocyte markers in the CD45.2 mouse peripheral blood.

FIG. 23 shows there are at least 25 GFP positive cells (green) in the converted HS/PCs transfected with pLenti-GFP in culture under UL™-TFK medium condition on day 3.

FIG. 24 shows only 1 GFP positive cell shown in the converted HS/PCs transfected with pLenti-GFP culture in the regular medium condition on day 3.

FIG. 25 shows a schematic view of an automatic multifunctional single cell processing and culture system, with door close in the upper panel and door open in the lower panel.

FIG. 26 shows a front and a side view (upper panel) and a schematic view (lower panel) of a fluid volume dispenser system of the cartridge-based dispensing system of the automatic multifunctional single cell processing and culture system.

FIG. 27 shows a lateral and a schematic view of a tissue grinder system of the automatic multifunctional single cell processing and culture system.

FIG. 28 shows a schematic view, a lateral cross view, and an explosive view of a vial equipped with a filter screen and grinder plates.

FIG. 29 shows a cross section view of the tissue grinder system of the automatic multifunctional single cell processing and culture system.

FIG. 30 shows a cross section view of the tissue grinder system in a lowered position (left panel) and a raised panel (right panel).

FIG. 31 shows a front view of arrangement of a cartridge-based dispensing system of the automatic multifunctional single cell processing and culture system.

FIG. 32 shows a schematic view of a cellular flask rotator. Within the Cellular Flask Rotator is an array of neodymium magnets that activate the magnetic nanoparticles as the fluid moves within the cellular flask.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the term “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “therapy” refers to any protocol, method, and/or agent that can be used in the management, treatment, and/or amelioration of a given disease, or a symptom related thereto. In certain embodiments, the terms “therapies” and “therapy” refer to a biological therapy, supportive therapy, and/or other therapies known to one of skill in the art, such as medical personnel, useful in the management or treatment of a given disease, or symptom related thereto.

As used in the disclosure, “treat”, “treatment”, and “treating” refer to the reduction or amelioration of the progression, severity, and/or duration of a given disease resulting from the administration of one or more therapies (including, but not limited to, the administration of microspheres disclosed herein).

Present system described herein features innovative strategies and sources for deriving HS/PCs, in particular, an innovative system and/or method for converting Ad MSCs to HS/PCs and differentiating into blood cells.

This innovation system of the present invention discloses that MSCs derived from mature adipocytes can be converted to HS/PCs and then differentiated into multiple peripheral blood cell types in vitro and in vivo. In one embodiment, the converted hematopoietic stem cells (HSCs) demonstrate a high gene transfection efficiency. This system of the present invention establishes a new source for autologous cell and gene therapy with less side effects than existing technologies.

The present invention discloses a system having four major breakthroughs.

In one embodiment, the present invention discloses unique Ad MScs cell culture protocols/reagents which convert Ad MSCs to HS/PCs in vitro. In one embodiment, the present invention creates conversions and expansion system for converting Ad MSCs into HS/PCs without introducing in any exogenous genes in vitro. In one embodiment, the conversion efficiency to HS/PCs is about 11.7%. In one embodiment, the present invention expands converted HS/PCs in large quantities in vitro.

In one embodiment, the present invention discloses unique protocols/reagents for differentiating the converted HS/PCs into various lineages of peripheral blood cells, both in vitro and in vivo. The present invention creates a system for differentiating the converted HS/PCs to various peripheral blood cells such as RBC, platelets, and WBC in vitro and on vivo.

In one embodiment, the present invention discloses unique protocols/reagents for enhancing system of gene transfection of the converted HS/PCs in vitro. The present invention creates a system enhancing gene delivery, and thus improves gene transfection efficiency of the converted HS/PCs. In one embodiment, the gene transfection efficiency of the present invention is 25-fold higher than regular reagents of existing technology. In one embodiment, the converted HS/PCs are gene carry-on cells, which provides guarantee for the success of gene therapy.

In one embodiment, the present invention discloses an automatic and bedside single cell isolation system having one or more of the following features (1) all-in-one integrated device of tissue grinding, enzyme digestion and nano-purification, centrifugation-free, contamination-free, with one scan recording system for rapid tissue single cells and specific blood cell isolation; (2) standardized protocol ensuring high quality of isolated cells; (3) bedside operation to minimize the time exposing to the environment and maximal cell culture survival rate.

In one embodiment, the present invention of converting Ad MSCs as a new course to derive HS/PCs in precision cell and gene therapy has one or more of the following advantages and implications.

In one embodiment, the present invention ensures the safety of the derived HS/PCs and blood cells produced therefrom. In particular, this innovation system does not require the introduction of any exogenous genes and thus there is no concern about the simultaneous emergence contamination of tumorigenic stem cells, as other induced or embryonic cells do. Also, Ad MSCs-converted HS/PCs are derived from autologous adipose tissues and autologous cell transplantation has no risk for GVHD side effects.

In one embodiment, the present invention is resource friendly. In particular, Ad MSCs are derived from adipose tissues (Ad), which is widely distributed, and widely available, as compared to MB-MS which is limited in resource: M: 10-20%, F: 20-30% in body. Thus the conversion rate is about 11.7% for Ad MSCs vs 1% in BM.

In one embodiment, the present invention is process efficient. In particular, adipose tissue collection is convenient and quick, which can usually be obtained through liposuction, minimally invasive surgery or other surgical procedures. All of which are less invasive and often results in fewer complications compared to the collection of bone marrow.

In one embodiment, the present invention is cultivation friendly. In particular, in vitro culture of Ad MSCs and converted HS/PCs can be accomplished efficiently in large scales for potential clinical needs. Ad MSCs have a long shelf life as compared with other types of stem cells, such as induced pluripotent or embryonic SCs.

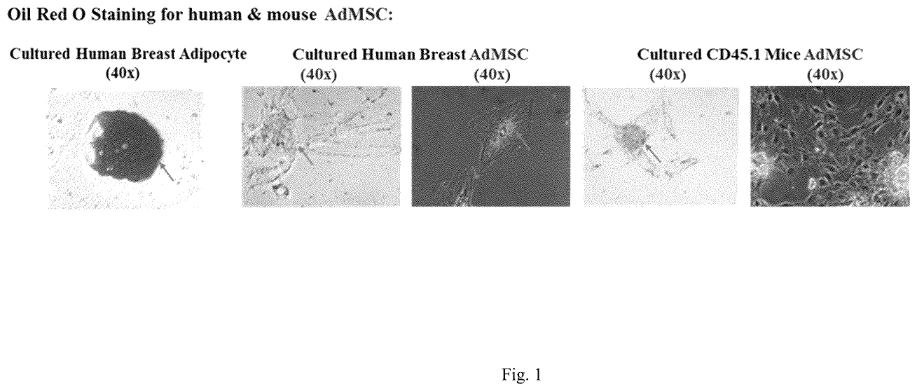

One important advantage of the present invention is that the conversion of Ad MSCs into HS/PCs and differentiation of HS/PCs into various blood cells are accomplished without co-culture with bone marrow derived HSCs and/or no exogenous genes inserted. That is, the HS/PCs are produced using a pure Ad MSCs cell line, and various blood cells are differentiated from the HS/PCs produced from the pure Ad MSCs cell line. No other cell, e.g. SVF, bone marrow derived HSCs, and etc., has ever been used for co-culture with the Ad MSCs and/or the HS/PCs produced therefrom. A pure Ad MSCs cell line is defined as a cell line consists essentially or only Ad MSCs. In practice, as shown in FIG. 1, a pure Ad MSCs cell line (Oil Red O Staining is a special staining for adipose derived mesenchymal stem cells (Ad MSC)) is obtained from culturing of biological cells obtained from a subject.

In one embodiment, the biological cells are obtained bedside, e.g., in adjacent to the subject from whom the sample is obtained.

In one embodiment, the present invention is ethics compliance. In particular, the present invention does not have the baggage of ethical controversy that embryonic stem cells may have in some contexts.

In one embodiment, the present invention is functionality-based. In particular, the present invention has strong value-added abilities and can secrete about 247 kinds of beneficial cell nutrition and stimulating factors, including anti-inflammatory agents, immune regulatory agents, angiogenesis agents, and nutritive agents. All of which are beneficial in the context of intensive treatment and regeneration.

In one embodiment, the present invention is application-based. In particular, the present invention has a wide differentiation potential, including gene carry on cells, bone cells, fat cells, nerve cells, vascular endothelial cells, cardiomyocytes, pancreatic cells, hepatocytes, HSCs, progenitor cells, and blood cells. Therefore, the present invention can be used in the regeneration of a variety of organs and tissues.

### Example 1—Automatic Multifunctional Single Cell Processing and Culture System

In one embodiment, the present invention discloses an all-in-one of multifunctional single cell processing and culture system. The system ensures a high efficiency of cell isolation and a high survival rate of the cells, while reduces costs of cell therapy for hospital and patients.

In one embodiment, the system provides a one-step isolation of Ad MSCs or tissues and immune cells without centrifugation, multi-steps, and risk of contaminations in about 60 minutes.

In one embodiment, the system provides a fully enclosed sterile constant temperature system, which ensures contamination free and a high cell survival rate.

In one embodiment, the system provides a standardized operation, as well as one scan data record, which ensure confidentiality & long-term tracking of the data.

In one embodiment, the system is portable, and support bedside use, which ensures widespread application.

In one embodiment, the system overcomes current challenges in cell processing, by maximizing cell survival rate, minimizing cell contamination, reducing cost. The system provides rapid, portable, and wide application for enhancing the effectiveness and accessibility of cell-based treatments.

In one embodiment, a machine for separating viable cells from tissue or blood samples includes one or more of the following components: an intake port which is designed to receive tissue samples or surgically removed tissue directly from a syringe, ensuring minimal handling and reducing contamination risk; a tissue grinder tube and/or a robotic rotary oscillator (Part A) which facilitates an efficient separation of viable cells from the tissue; a cell culture flask (Part A-b) containing about 5-10 ml nanoparticle-specific antibody solution; a robotic pipetting arm of nozzles (Part B) that dispense the cell suspension and other solution, which performs cell transfer and washes; a shakable magnetic platform (Part C), which facilitates cell attachment to the bottom of the cell culture flask or conjugation with specific nano-antibodies; a robotic capper arm (Part D) to seal the flask, which facilitates sterile transportation of the cell culture flask; an automatic or semi-automatic lever connector mechanism (Part E) which connects all post-disinfection solution cartridges in the sealed container to maintain sterility and prevent direct human handling; an insulated carrier (Part F) for the sealed cell culture flask, equipped with temperature control at 37° C. and CO₂ supplementation, increasing the number of viable cells from transportation from patient bedside operation to lab for further cell processing (The machine should remain at a constant 37° C. when the power is on); multiple germicidal UV+ Ozone (Part G) is placed throughout the system, ensuring sterility within the system; and a bar code scanner (Part H) for registering the tissue sample into the machine before processing, linking the sample with a tagged cell culture flask for traceability and compliance with medical documentation practices.

In one embodiment, an operator fills the grinder vial with a tissue sample then places the vial onto the bottom adapter of the grinder subassembly.

In one embodiment, the operator then manipulates the over center mechanism to raise the grinder vial towards the grinder gearbox.

In one embodiment, the bottom and top seals of the grinder are punctured, coupling the vial to the machine.

In one embodiment, 5 ml of buffer agent is dispensed into the grinder to facilitate separation of cells from the tissue.

In one embodiment, after a set amount of time, another 5 ml of buffer agent is dispensed into the grinder and negative pressure is applied by the peristaltic pump with the outlet of the pump going towards the cellular flask.

In one embodiment, within the cellular flask, the isolated cells are washed with saline then have magnetic nanoparticles added.

In one embodiment, after several washings, a buffer agent is applied to the cellular flask before the flask is sealed ready for transportation.

In one embodiment, the present invention discloses a small fluid volume dispenser system. The purpose of the system is to dispense small volumes of fluid, specifically, between 5-10 ml, in a sterile manner. This is crucial for tasks requiring precision and cleanliness, such as those in cellular biology or medical research.

In one embodiment, the present invention discloses a cartridge-based dispensing process. Instead of using pumps, tubing, or reservoirs, which could compromise sterility, the system uses pre-filled cartridges. Each cartridge contains a precise amount of fluid. A set of needles punctures the cartridge both at the top and bottom to facilitate fluid flow. The top needle is actuated to create a vent hole as well as push the cartridge towards a bottom needle for draining.

In one embodiment, the present invention discloses hydrophobic funnel. In particular, as fluid is dispensed, it passes through a hydrophobic funnel, which repels water, ensuring that the fluid flows smoothly into the desired container without contamination or loss of product.

The advantages of the system include eliminating the need for measurement sensors and positive displacement pumps, simplifying the system; and reducing loss of cell buffer agent, important for minimizing waste and cleanup.

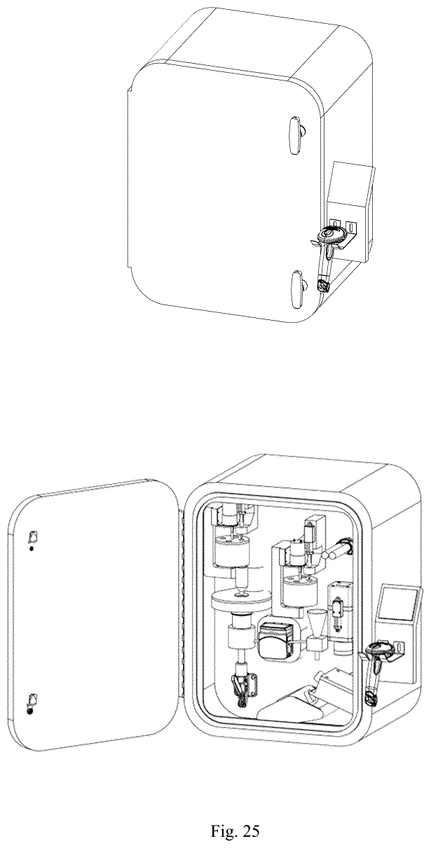

FIG. 25 shows a schematic view of an automatic multifunctional single cell processing and culture system, with door close in the upper panel and door open in the lower panel.

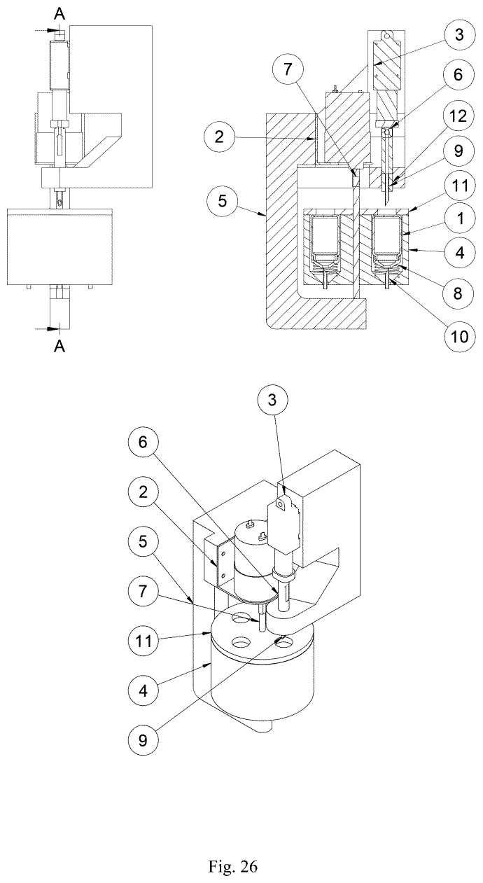

FIG. 26 shows a front and a side view (upper panel) and a schematic view (lower panel) of a fluid volume dispenser system of the cartridge-based dispensing system of the automatic multifunctional single cell processing and culture system.

In particular, one or more cartridge 1 is disposed inside a cylinder 4. A spring may be disposed below the cartridge to push the cartridge upward against a cylinder cap 11 disposed on top of the cylinder. The cartridge 1 may be connected to a bottom drain needle 10 on its bottom. A cylinder rod 7 passed trough the cylinder 1 in the center. A motor 2 is supported by a bracket 5 and disposed above the cylinder 4. The motor 2 is connected to the cylinder rod 7 and drives it. Above the cartridge 1, a linear actuator 3 is connected to a needle extension, which receives a top vent needle 9. In one embodiment, there is a needle holder 12 in direct contact with the vent needle 9.

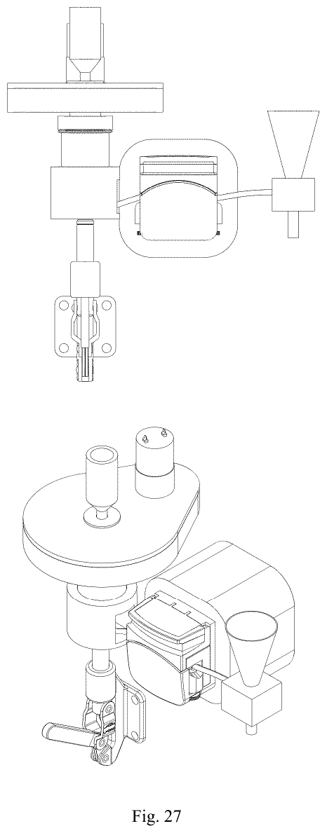

FIG. 27 shows a lateral and a schematic view of a tissue grinder system of the automatic multifunctional single cell processing and culture system.

In particular, in one embodiment, the tissue grinder system includes a 30 ml vial equipped with a 70 nm filter screen and grinder plates. During the operation, the tissue samples are placed inside the vial, between a set of grinder plates. The system is sealed with a screw cap, compressing the tissue in between the two grinder plates.

For a fluid dispensing for grinding, 5 ml of buffer agent is dispensed into the vial to aid the grinding. This is done within the cell isolator machine, ensuring sterility.

In one embodiment, the vial is placed on an adapter and lifted to engage with a gear that drives the grinding action. A needle funnel, which receives dispensed fluid, also punctures a seal for sterility.

In one embodiment, in a post-grinding process, after grinding, another 5 ml of fluid is added to rinse the sample.

In one embodiment, a peristaltic pump applies negative pressure to collect the suspended cells, which are then transferred into a cellular flask for further processing, maintaining a sterile environment throughout.

This system aims to process biological samples in a sterile, efficient manner, minimizing the risk of contamination and ensuring the integrity of the samples for research or medical purposes.

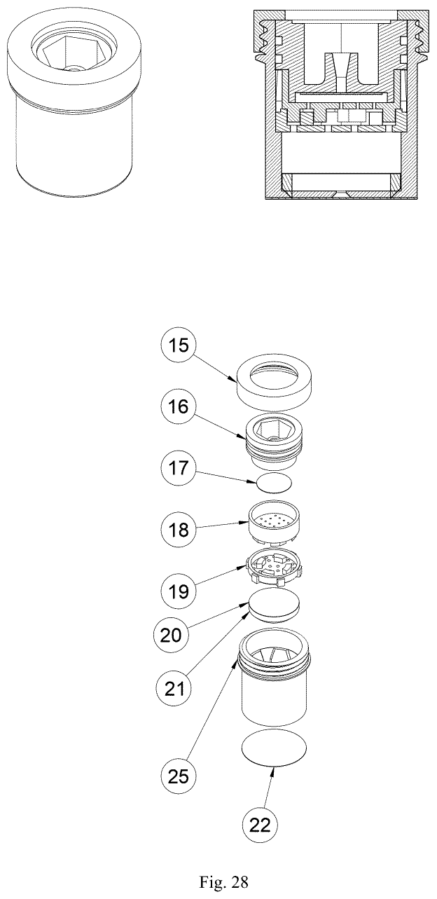

FIG. 28 shows a schematic view, a lateral cross view, and an explosive view of a vial equipped with a filter screen and grinder plates.

In one embodiment, the vail includes a screw cap 15 receiving a plug 16. A top induction seal 17 is received by the plug 16. A top grinder plate 18 and a bottom grinder plate 19 pairs with each other. A filter screen 20 inside a filter holder 21 is disposed below the top grinder plate 18 and the bottom grinder plate 19. A vial 25 matches with the screw cap 15 and form a housing, which houses the aforementioned parts. A bottom induction seal 22 is attached to the bottom of the vial 25.

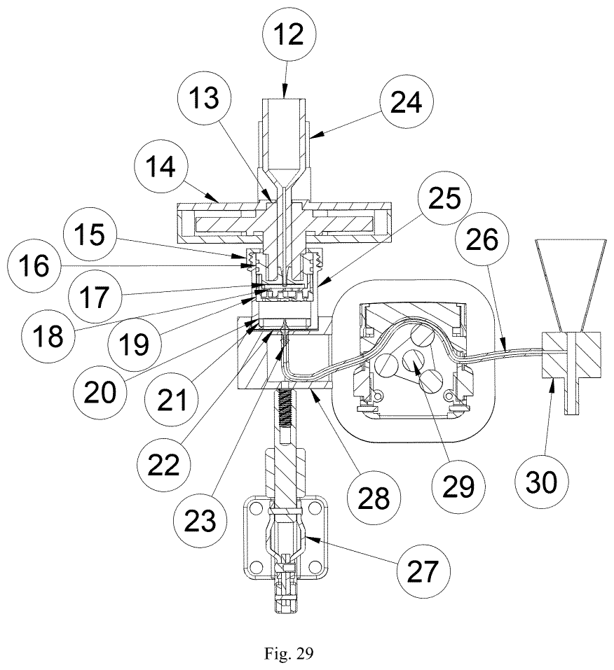

FIG. 29 shows a cross section view of the tissue grinder system of the automatic multifunctional single cell processing and culture system.

In one embodiment, the tissue grinder system includes a needle funnel 12 with its bottom tube passing through a through hole large gear 13 disposed inside a gearbox. The bottom end of the needle funnel 12 is received inside the vial described above. An induction seal needle drain 23 is fluidly connected to the bottom of the vial 25, and transport the fluid through a silicon tubing. The silicon tubing 26 is received by an over center mechanism 28 supported by a bottom adapter 27. The silicon tubing 26 then passes through a peristaltic pump 29, and fluidly connected to a dispense funnel 30.

In one embodiment, the fluid volume dispenser system is arranged above the tissue grinder system in the device. Thus, a solution, e.g., a buffering agent, is dispensed by the fluid volume dispenser system and dispensed into the vial 25, through the top of the vial screw cap 15, and right through the coupling that directs power to the top and bottom grinder plates 18/19.

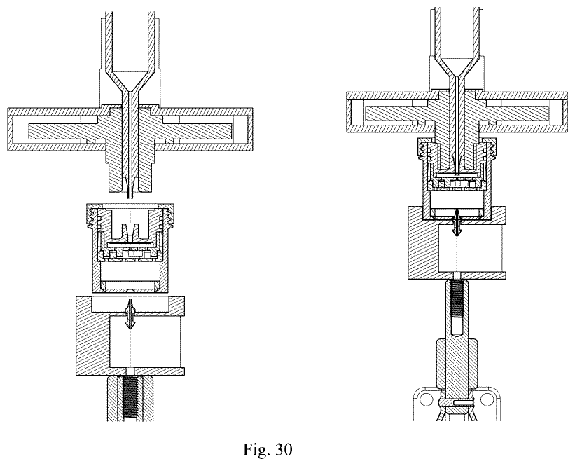

FIG. 30 shows a cross section view of the tissue grinder system in a lowered position (left panel) and a raised panel (right panel).

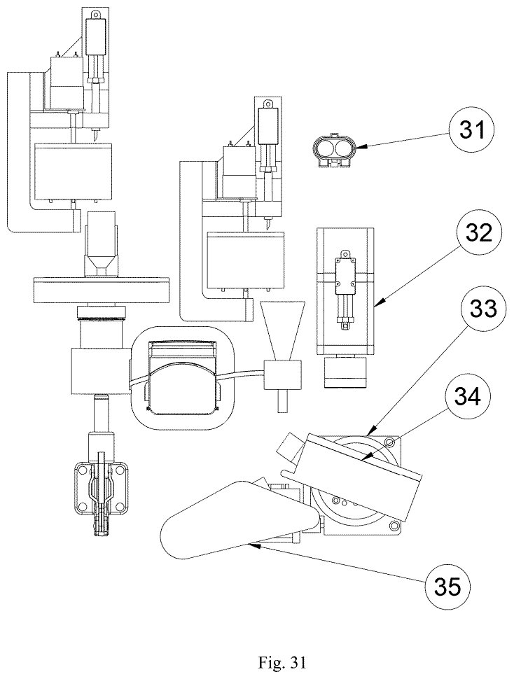

FIG. 31 shows a front view of arrangement of a cartridge-based dispensing system of the automatic multifunctional single cell processing and culture system. In particular, except for a fluid volume dispenser system placed above the tissue grinding system, in one embodiment, the system also includes one or more of a UV Disinfection Light 31, a capper 32, a cellular flask rotator 33 configured to receive a cellular flask 34, and fluidly connected to a drain 35.

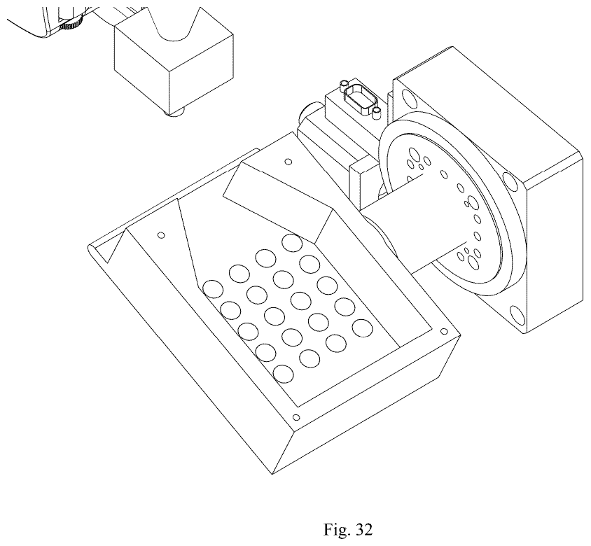

FIG. 32 shows a schematic view of a cellular flask rotator. Within the Cellular Flask Rotator is an array of neodymium magnets that activate the magnetic nanoparticles as the fluid moves within the cellular flask.

### Example 2—Protocols for Tissue Single Cells Isolation and Ad MSCs Culture Protocol for Preparing Tissue

In one embodiment, the present invention discloses a protocol for preparing tissue. The protocol should be modified accordingly based on the weight and/or volume of the tissue being prepared without deviating from the essence of the present invention.
1. To prepare 37° C. prewarm wash buffer 500 ml, add 5.5 ml 100× Antibiotic-Antimycotic Liquid (AA) into 500 ml wash buffer.
2. To prepare completely Mesenchymal Stem Cell Expansion medium: Add serum-supplemented B into 500 mL bottle of Mesenchymal Stem Cell Expansion Medium A (UL™-MSC-E) and 5 ml 100× Antibiotic-Antimycotic Liquid (AA).
3. Place an absorbent underpaid in the hood.
4. To a sterile 100 mm culture dish, add ˜10 mL of prewarmed wash buffer prepared in Step 1.
5. Obtain the tissue and place the container with the tissue in the laminar flow hood.
6. Remove the lid from the 100 mm dish and using sterile forceps transfer the tissue to the prewarmed wash buffer dish.
7. Wash the tissue by agitating with forceps in the medium contained in the 100 mm dish for two or three times.

### Protocol for Primary Mature Fat or Other Tissue Isolation

In one embodiment, the present invention discloses a protocol for primary mature fat or other tissue isolation.
1. Directly place the tissue from surgically removed in Part A of the system in Example 1. Part A automatically grinds tissue into single cells by Robotic Rotary Oscillator. After breaking up the tissue, the grinder tube automatically turns into a vertical position. The cells suspension will flow down to the bottom of the grinder tube under gravity. Then, wash the grinder with 10 ml UL™-TMSCs-IS.
2. Incubate the cells suspension collected for 20 to 30 minutes at 37° C. with shake. Transfer all cells suspension into a new cell culture flask.
3. Place the new cell culture flask flat and stand the flask at 37° C. for 10 minutes on Part C.
4. Absorb the supernatant by Part B of the system, and then add 10 ml wash buffer into the flask to wash the cells 2 times. Add 10-20 ml UL™-TMSCs-ISMSC-E (depends on size of the flask) to flask and seal the culture flask by Part D.
5. Transfer the culture flask into incubator at 37° C. with 5% CO₂ and incubate for 5-7 days.

### Protocol for Isolating and Culture Ad MSCs

In one embodiment, the present invention discloses a protocol for isolating and culture Ad MSCs.
1. Using sterile forceps to place and flatten the tissue onto the grinder.
2. Transfer the grinder to cell processor.
3. Start program 1 for fat or other tissue.
4. Take flask from processor and transfer flask into the incubator and culture at 37° C. and 5% CO2 with a proper humidly.

Results: 72 hours after seeding primary cultures, observe the cultures using phase contrast microscopy. Some AdMSCs attached to the surface of the flask are observed. The medium should be changed twice a week.

Table 1 shows a general process for troubleshooting for the protocol for isolating and culture Ad MSCs.

TABLE 1
General process for troubleshooting for the
protocol for isolating and culture Ad MSCs

| Problem | Cause | Solution |
|---|---|---|
| Cells attach | Tissue stored at | Use tissue within 24 hours of |
| poorly | lower degree | harvest for the best results. |
| | or too long/ | Store tissue in culture medium |
| | improperly. | at 37° C. until use. |
| Cells grow | Medium and/ | Check the expiration date on the label |
| slowly | or supplement | of the products and do not use the |
| | stored | product after the expiration date. |
| | incorrectly, | Check the storage conditions as |
| | beyond | described in the product manual. |
| | expiration date | Confirm that the |
| | | products were stored properly. |
| | Supplemented | Store supplemented medium at |
| | medium stored | 4° C. for up to 1 month from the |
| | too long or | time the basal medium is |
| | improperly | supplemented with reagent B. |
| Cells become | Improper | Store tissue in medium containing |
| contaminated | tissue | antibiotic/antimycotic. Store |
| with | storage | tissue at 37° C. Wash |
| micro- | | tissue thoroughly in medium |
| organisms | | containing antibiotic/antimycotic |
| | | at the start of the procedure. |
| | Expired or | Check the expiration date |
| | incorrect | on the product and do not |
| | concentration | use after the expiration date. |
| | of antibiotic/ | Check for the proper dilution of the |
| | antimycotic | product int he supplemented |
| | solution used | medium and correct if necessary. |

### Protocol for Peripheral Blood Cells Isolation

In one embodiment, the present invention discloses a protocol for peripheral blood cells isolation.
1. Inject Buff y coat into Part A-b with nanoparticle-specific antibody reagent. Rocker and incubate for 15 to 30 minutes at 37° C. Place Part A-b on Part C for 2-5 minutes. Absorb the supernatant by Part B, and then add 10 ml wash buffer into the Part A-b flask by Part B, repeat 2 times. Release Part A-b from Part C. Add 10 ml culture medium B+D into Part A-b and seal Part A-b by Part D.
2. Transfer the Part A-b into cell culture incubator at 37° C. with 5% CO2 for 5-7 days.
   Protocols for Converting Ad MSCs into HS/PCs:

After initial seeding of the primary Ad MSCs culture, once the cells are ˜90% confluent (7-13 days), subculture the Ad MSCs by using UL™-Hematopoietic Stem Cell (HS/PC) Converting kit (UL™-HS/PC-C kit) disclosed in Example 2.

This protocol is designed for the subculture of one 60 mm culture dish or 25 cm² culture flask of actively proliferating cells near confluence. If different-sized culture vessels are to be used, adjust the reagent volumes accordingly.

In one embodiment, the reagents should not be warmed prior to use.
1. Prepare UL™-HS/PC-C completely medium: Dissolve reagent B (Nutrition supplement) and C (Converting supplements) on ice. When the reagents are completely dissolved, carefully pipetting reagent into the reagent A bottle and mix well.
2. Remove all the culture medium from the flask.
3. Wash cells with 5 mL Ca++ and Mg++-free PBS to the flask. Rock the flask to ensure that the entire surface is covered.
4. Immediately remove the PBS solution from the flask and add 5 mL (for 60 mm Dish), 8 ml (for 25 cm² Flask) UL™-HS/PC-C completely medium.
5. Incubate the flask at 37° C. incubator in 5% CO2 with humidity for 3-6 days. Medium is changed about every 3 days.

### Protocols for Subculture of Converted HS/PCs.

1. Decant media from cell culture dish or flask. Rinse cells with 5 ml of Dulbecco's Phosphate Buffered Saline (D-PBS) without calcium and without magnesium. Decant D-PBS.
2. Add an appropriate 1 ml in 60 mm dish or 2 ml in a 75 cm² flask of prewarmed Trypsin-EDTA (0.25%) to dish or flask.
3. Incubate at 37° C. until cells have detached (observe at 2-minute intervals).
4. Add 2 to 4 ml of cell culture growth media and transfer cell suspension to two or more new 100 mm Dish or 75 cm2 Flask.
5. After 48-hour culture, continue culture the cell in UL™-HS/PC-C completely medium but without the SB431542, CHIR99021 and Y-27632 for 4 days.
6. Subculture the converted HS/PCs or Cryopreservation of the converted HS/PCs.

In one embodiment, if there is no need to use the converted HS/PCs for future study, pipetting the cells into a new 15 ml tube. Centrifuge for 5 to 10 minutes at 100×g. Discard supernatant and suspend cell pellet with reagent of your purpose.

### Protocol for Cryopreservation of Converted HS/PCs.

If a surplus of cells is available from subculturing, they should be treated with the appropriate protective agent (e.g., DMSO or glycerol) and stored at temperatures below −130° C. (cryopreservation) until they are needed.

View the culture under a microscope to ascertain the condition of the culture (i.e., confluence, mitotic activity). In one embodiment, the present invention cryopreserves converted HS/PCs when the culture is approximately 90% confluent and actively growing.

Warming the reagents prior to use is not recommended.

### Follow Steps 1-4 in Subculture of Converted HS/PCs.

1. Resuspend the cell pellet in 2-3 ml of cold (4° C.) UL™-Cryo medium and transfer 1 ml (to yield approximately 2-5×106 cells/m) to CryoFlex Tubing
2. Determine the number of viable cells/mL using a hemocytometer and dilute to the desired final cell density (5-10×106 viable cells/mL is recommended).
3. Cryopreserve the cells using a controlled-rate freezer or other appropriate device, then transfer to liquid nitrogen storage (vapor phase).

Results: Twenty-four hours after converting reagent in cultures, observe the cultures using phase contrast microscopy. In one embodiment, some cells are detached to the medium of the flask although there will be some floating cells and debris. After 72 hours, carefully change the medium so that one does not dislodge lightly adherent cells. Converted the cells will continue grows until 85 to 90% confluent.

Table 2 shows a troubleshooting chart of cryopreservation of Converted HS/PCs.

TABLE 2
Troubleshooting chart of cryopreservation of Converted HS/PCs

| Problem | Cause | Solution |
|---|---|---|
| Cells | Ad MSCs stored too | Before freezing Ad MSCs, check |
| attach | long/improperly. | the cell viability and try to recover |
| poorly | | cells as quickly as possible. |
| | | Culture medium prewarn at 37° C. |
| | | until use. |
| Cells | Medium and/or | Check the expiration date on the |
| grow | supplement stored | label of the products and do not use |
| slowly | incorrectly, beyond | the product after the expiration |
| | expiration date | date. |
| | | Check the storage conditions as |
| | | described in the product manual. |
| | | Confirm that the products were |
| | | stored properly. |
| | Supplemented | Store supplemented medium at 4° C. |
| | medium stored too | for up to 1 month from the time the |
| | long or improperly | basal medium is supplemented with |
| | | reagent A. |

Protocols for Differentiation RBC and Platelets from the Converted HS/PCs In Vitro.

This protocol is designed for the subculture of one 60 mm culture dish or 25 cm² culture flask of actively proliferating cells near confluence. If different-sized culture vessels are to be used, adjust the reagent volumes accordingly.

It is not recommended to change medium between day 10 to 25. In one embodiment, it is recommended to add fresh medium twice a week.

Prepare UL™-RBC/Platelets completely medium: Dissolve reagent B (nutrition supplement) and C (RBC & Platelets supplements) on ice. When the reagents are completely dissolved, carefully pipetting reagent into the reagent A bottle and mix well.
1. After initial seeding of the converted HS/PCs in 60 mm culture dish or 25 cm flask, once the cells are ˜90% confluent (7-13 days), remove all the culture medium from the dish or flask. And then add 5 ml (60 mm Dish), 10 ml (25 cm² Flask) Red Blood Cell and Platelets completely medium (UL™-h or m RBC kit) into culture dish or Flask.
2. Add the 8 ml RBC/Platelets completely medium into to cell culture dish or Flask twice a week.
3. At day 18-25, collect the supernatant containing suspended cells into a new 50 ml centrifuge tube.
4. Spin down the cells at 1000 rpm for 10 minutes, Wash cells with 10 mL Ca++ and Mg++-free PBS for 2 or 3 times.
5. Keep cell pellets for future use.

Results: On cultures day 18 to 21, one can see little dark color small round cells floating in the supernatant.

Table 3 shows troubleshooting process for differentiation RBC and Platelets from the converted HS/PCs in vitro.

TABLE 3
Troubleshooting process for differentiation RBC and
Platelets from the converted HS/PCs in vitro

| Problem | Cause | Solution |
|---|---|---|
| Cells | stored too | Before freezing converted HS/PCs, |
| poorly | long/improperly. | check the cell viability and try to |
| | | recover cells as quickly as possible. |
| | | Culture medium prewarn at 37° C. |
| | | until use |
| Cells | Medium and/or | Check the expiration date on the label |
| grow | supplement | of the products and do not use the |
| slowly | stored | product after the expiration date. |
| | incorrectly, | Check the storage conditions as |
| | beyond | described in the product manual. |
| | expiration date | Confirm that the products were stored |
| | | properly. |
| | Supplemented | Store supplemented medium at 4° C. for |
| | medium stored | up to 1 month from the time the basal |
| | too long or | medium is supplemented with reagent |
| | improperly | A. |

Protocols for Differentiation WBC from Converted HS/PCs In Vitro

This protocol is designed for the subculture of one 60 mm culture dish or 25 cm² culture flask of actively proliferating cells near confluence. If different-sized culture vessels are to be used, adjust the reagent volumes accordingly.

It is not recommended to change medium between day 10 to 35. In one embodiment, it is recommended to add fresh medium twice a week.

Prepare UL™-WBC completely medium: Dissolve reagent B (Basic nutrition supplement) and C (WBC supplements) on ice. When the reagents are completely dissolved, carefully pipet the B & C reagent into the reagent A bottle and mix well.
1. After initial seeding of the converted HS/PCs in 60 mm culture dish or 25 cm² flask, once the cells are ˜90% confluent (7-13 days), remove all the culture medium from the dish or flask. And then add 5 ml (60 mm Dish), 10 ml (25 cm² flask) White cells completely medium (UL™-h or m WBC kit) into culture dish or Flask.
2. Add the 8 ml WBC completely medium into to cell culture dish or Flask twice a week.
3. At day 20-35, collect the supernatant containing suspended cells into a new 50 ml centrifuge tube.
4. Spin down the cells at 1000 rpm for 10 minutes, Wash cells with 10 mL Ca++ and Mg++-free PBS for 2 or 3 times.
5. Keep cell pellets for future use.

Results: On cultures day 10 to 35, one can see little dark color small round cells floating in the supernatant.

Table 4 shows troubleshooting process for differentiation WBC from converted HS/PCs in vitro.

TABLE 4
Troubleshooting process for differentiation
WBC from converted HS/PCs in vitro

| Problem | Cause | Solution |
|---|---|---|
| Cells | stored too | Before freezing converted HS/PCs, check |
| poorly | long/ | the cell viability and try to recover |
| | improperly. | cells as quickly as possible. Culture |
| | | medium prewarn at 37° C. until use |
| Cells | Medium and/ | Check the expiration date on the label |
| grow | or supplement | of the products and do not use the |
| slowly | stored | product after the expiration date. |
| | incorrectly, | Check the storage conditions as |
| | beyond | described in the product manual. |
| | expiration date | Confirm that the |
| | | products were stored properly. |
| | Supplemented | Store supplemented medium |
| | medium stored | at 4° C. for up to 1 month |
| | too long or | from the time the basal medium is |
| | improperly | supplemented with reagent A. |

### Protocol for Enhancing Gene Transfection Efficiency in Converted HS/PCs as a Gene Carry-on Cells In Vitro.

This protocol is designed for the subculture of one 60 mm culture dish of actively proliferating cells near confluence. If different-sized culture vessels are to be used, adjust the reagent volumes accordingly.

It is not recommended that using UL™-Gene transfection enhancer Kit (UL™-h or m TFK) after 24-hour transfection.
1. Prepare UL™-Gene transfection enhancer Kit (UL™-h or m TFK) completely medium: Dissolve reagent B (nutrition supplement) and C (Gene transfection enhancer supplements) of the UL™-Human or Mouse Gene transfection enhancer Kit (UL™-h or m TFK) on ice. When the reagents are completely dissolved, carefully pipetting reagent into the reagent A bottle and mix well.
2. After regular transfection 24-hours, remove all the culture medium from the dish. And then add 5 ml (60 mm Dish), UL™-Gene transfection enhancer completely medium (UL™-h or m TFK kit) into culture dish.
3. Change TFK medium twice a week. When the cells near confluence, harvest cells to sorting the positive cells.
4. Culture the pure positive cell with TFK completely medium for another 3-6 days, change culture medium to basic cell culture medium A with nutrition supplement B. Change the medium twice per week until cells grow to enough cells for gene therapy.

Results: After transfection day 6, there should be more than 70% positive cells (if there is the visible marker in the system).

Table 5 shows troubleshooting for enhancing gene transfection efficiency in converted HS/PCs as a gene carry-on cells in vitro.

TABLE 5
Troubleshooting for enhancing gene transfection efficiency
in converted HS/PCs as a gene carry-on cells in vitro

| Problem | Cause | Solution |
|---|---|---|
| Cells | Converted | Before freezing converted HS/PCs, |
| | HS/PCs stored | check the cell viability and try to |
| | too | recover cells as quickly as possible. |
| poorly | long/improperly. | Culture medium prewarn at 37° C. |
| | | until use |
| Cells grow | Medium and/or | Check the expiration date on the |
| slowly | supplement | label of the products and do not use |
| | stored | the product after the expiration date. |
| | incorrectly, | Check the storage conditions as |
| | beyond | described in the product manual. |
| | expiration date | Confirm that the products were |
| | | stored properly. |
| Low | Supplemented | Store supplemented medium at 4° C. |
| transfection | medium stored | for up to 1 month from the time the |
| efficiency | too long or | basal medium is supplemented with |
| | improperly | reagent A. |

### Example 3—Reagents and Kits for Tissue Single Cells Isolation and Ad MSCs Culture

In one embodiment, an enzyme digestive reagent (UL™-Tissues MSCs Isolation—UL™-TMSCs-IS kit) includes 1×PBS with 0.25% Collagenase Type I/5 mM glucose/1.5% BSA.

In one embodiment, the nanoparticle-specific antibody reagent includes <100 nm Lipid-based Nanoparticle (LNPs) or Polymer-based (PNPs) with specific antibody depending on customer's purpose.

### 1. UL™-Mesenchymal Stem Cell Expansion Medium (UL™-MSC-E)

In one embodiment, the present invention discloses cell culture reagents for Ad MSCs and blood cell culture UL™-Mesenchymal Stem Cell Expansion Medium (UL™-MSC-E).

In one embodiment, UL™-Mesenchymal Stem Cell Expansion Medium (UL™-MSC-E) includes one or more of the flowing four components.
A. Basic Ad MSCs (adherent cells) culture medium (Store at 4° C.): 500 ml of High glucose DMEM/F12 with L-Glutamine and 2.438 g/L Sodium Bicarbonate, Penicillin-Streptomycin (10,000 U/ml), 1% NEAA. 1% HEPES.
B. Basic nutrition supplement (Store at −20° C.): 25 ml Fetal bovine serum (10%) and 25 ml Horse serum (10%) (Both of sera need Heat Inactivation).
C. Basic suspension cells culture medium (Store at 4° C.): 500 ml of high glucose RPMI 1640 with L-Glutamine and 2.438 g/L Sodium Bicarbonate, Penicillin-Streptomycin (10,000 U/ml), 1% NEAA. 1% HEPES.
D. Nutrition supplement (Store at −20° C.): 50 ml Fetal bovine serum (10%) (Heat Inactivation).

### 2. UL™-Human or Mouse HS/PCs Converting Kit (UL™-hHS/PC-C Kit)

In one embodiment, the present invention discloses reagents for converting Ad MScs to HS/PCs in vitro.

The UL™-Human or Mouse Hematopoietic Stem Cell (HS/PC) Converting kit (UL™-hHS/PC-C kit) includes one or more of the following three components.
A. Basic cell culture medium (Store at 4° C.): 500 ml of High glucose DMEM/F12 with L-Glutamine and 2.438 g/L Sodium Bicarbonate, Penicillin-Streptomycin (10,000 U/ml), 1% NEAA. 1% HEPES.
B. Basic nutrition supplement (Store at −20° C.): 25 ml Fetal bovine serum (10%) and 25 ml Horse serum (10%) (Both of sera need Heat Inactivation).
C. Converting supplements (Store at −80° C.) in Table 6.

TABLE 6
Converting supplements for
UL ™-Human or Mouse HS/PCs Converting kit

| Converting | | Final |
|---|---|---|
| supplements | Name | Concentration |
| 1 | Insulin | 10 ug/ml |
| 2 | Holo-Transferrin | 160 ug/ml |
| 3 | Sodium Selenite (ITS liquid) | 5 ng/ml |
| 4 | L-Ascorbic Acid | 50 ug/ml |
| | | (1 mM) |
| 5 | GM-CSF | (10 ng/ml) |
| 6 | SCF | 300 ng/ml |
| 7 | VEGF | 20 ng/ml |
| 8 | IGF-I | 40 ng/ml |
| 9 | IGF-II | 200 ng/ml |
| 10 | IL-3 | 5 ng/ml |
| 11 | Flt3-L | 50 ng/ml |
| 12 | Thrombopoietin (TPO) | 50 ng/ml |
| 13 | Dexamethasone | 1 uM |
| 14 | Fatty Acid free BSA | 1% |
| 15 | 1-Thioglycerol | 0.45 mM |
| 16 | SB431542 for 48 hr | 6 μM |
| 17 | CHIR99021 for 48 hr | 3 μM |
| 18 | Y-27632 for 48 hr | 10 μM |

3. UL™-Human or Mouse Red Blood Cell and Platelets Kit (UL™-h or m RBC Kit) for Differentiation of the Converted HS/PCs into RBC and Platelets

In one embodiment, the present invention discloses reagents for inducing differentiation of the converted HS/PCs into RBC and Platelets, in vitro and in vivo.

UL™-Human or Mouse Red Blood Cell and Platelets kit includes one or more of three following components.
A. Basic cell culture medium (Store at 4° C.): 500 ml of High glucose DMEM/F12 with L-Glutamine and 2.438 g/L Sodium Bicarbonate, Penicillin-Streptomycin (10,000 U/ml), 1% NEAA. 1% HEPES.
B. Basic nutrition supplement (Store at −20° C.): 50 ml Fetal bovine serum (10%) and 50 ml Horse serum (10%) (Both of sera need Heat Inactivation).
C. RBC & Platelets supplements (Store at −80° C.) are listed in Table 7.

TABLE 7
RBC & Platelets supplements for UL ™-Human or
Mouse Red Blood Cell and Platelets kit

| RBC & Platelets | | Final |
|---|---|---|
| supplements | Name | Concentration |
| 1 | Insulin | 10 ug/ml |
| 2 | Holo-Transferrin | 160 ug/ml |
| 3 | Sodium Selenite (ITS liquid) | 5 ng/ml |
| 4 | L-Ascorbic Acid | 50 ug/ml |
| | | (1 mM) |
| 5 | SCF | 300 ng/ml |
| 6 | VEGF | 20 ng/ml |
| 7 | IGF-I | 40 ng/ml |
| 8 | IGF-II | 200 ng/ml |
| 9 | IL-3 | 5 ng/ml |
| 10 | Flt3-L | 50 ng/ml |
| 11 | Thrombopoietin (TPO) | 50 ng/ml |
| 12 | Dexamethasone | 1 uM |
| 13 | Fatty Acid free BSA | 1% |
| 14 | 1-Thioglycerol | 0.45 mM |
| 15 | EPO | 5 ng/ml |
| 16 | Ferrous Sulfate | 900 ng/ml |
| 17 | PDGF BB | 100 ng/ml |
| 19 | Activin A | 100 ng/ml |

### 4. UL™-Human or Mouse White Blood Cell Kit (UL™-h or m WBC Kit)

In one embodiment, the present invention discloses reagents for differentiation of the converted HS/PCs into various lineages of peripheral WBC in vitro and in vivo. The UL™-Human or Mouse White Blood Cell kit (UL™-h or m WBC kit) includes one or more of the three following components.
A. Basic cell culture medium (Store at 4° C.): 500 ml of High glucose DMEM/F12 with L-Glutamine and 2.438 g/L Sodium Bicarbonate, Penicillin-Streptomycin (10,000 U/ml), 1% NEAA. 1% HEPES.
B. Basic nutrition supplement (Store at −20° C.): 50 ml Fetal bovine serum (10%) and 50 ml Horse serum (10%) (Both of sera need Heat Inactivation).
C. WBC supplements (Store at −80° C.) in Table 8.

TABLE 8
WBC supplements for UL ™-Human
or Mouse White Blood Cell kit

| WBC | | Final |
|---|---|---|
| supplements | Name | Concentration |
| 1 | Insulin | 10 ug/ml |
| 2 | Holo-Transferrin | 160 ug/ml |
| 3 | Sodium Selenite | 5 ng/ml |
| | (ITS liquid) | |
| 4 | L-Ascorbic Acid | 50 ug/ml |
| | | (1 mM) |
| 5 | SCF | 300 ng/ml |
| 6 | VEGF | 20 ng/ml |
| 7 | IGF-I | 40 ng/ml |
| 8 | IGF-II | 200 ng/ml |
| 9 | IL-3 | 5 ng/ml |
| 10 | Flt3-L | 50 ng/ml |
| 11 | Thrombopoietin | 50 ng/ml |
| | (TPO) | |
| 12 | Dexamethasone | 1 uM |
| 13 | Fatty Acid free BSA | 1% |
| 14 | 1-Thioglycerol | 0.45 mM |
| 15 | BMP4 | 10 ng/ml |
| 16 | IL-7 | 20 ng/ml/5 ng/ml/2 ng/ml |
| 17 | IL-12 | 10 ng/ml |
| 18 | IL-11 | 5 ng/ml |
| 19 | IL-6 | 10 ng/ml |
| 20 | IL-2 | 2 ng/ml |
| 21 | FGF-b | 10 ng/ml/5 ng/ml |
| 22 | TGF-a | 5 ng/ml |
| 23 | TGF-b1 | 5 ng/ml |
| 24 | TGF-b2 | 5 ng/ml |
| 25 | TGF-b3 | 5 ng/ml |
| 26 | IL-1a | 5 ng/ml |
| 27 | IL-1b | 5 ng/ml |
| 28 | IL-4 | 5 ng/ml |
| 29 | IL-5 | 5 ng/ml |
| 30 | IL-8 | 5 ng/ml |
| 31 | IL-10 | 5 ng/ml |
| 32 | IL-12 | 5 ng/ml |
| 33 | IL-32a | 5 ng/ml |
| 34 | mIL-36R2 | 5 ng/ml |
| 35 | GM-CSF | 10 ng/ml |

### 5. UL™-Human or Mouse Gene Transfection Enhancer Kit (UL™-h or m TFK)

In one embodiment, the present invention discloses an enhancing system for gene delivery of the converted HS/PCs in vitro. UL™-Human or Mouse Gene transfection enhancer Kit (UL™-h or m TFK) includes one or more of the three following components.
A. Basic cell culture medium (Store at 4° C.): 500 ml of High glucose DMEM/F12 with L-Glutamine and 2.438 g/L Sodium Bicarbonate, Penicillin-Streptomycin (10,000 U/ml), 1% NEAA. 1% HEPES.
B. Basic nutrition supplement (Store at −20° C.): 50 ml Fetal bovine serum (10%) and 50 ml Horse serum (10%) (Both of sera need Heat Inactivation).
C. Gene transfection enhancer supplements (Store at −80° C.) in Table 9.

TABLE 9
Gene transfection enhancer supplements for UL ™-
Human or Mouse Gene transfection enhancer Kit

| Gene | | |
|---|---|---|
| transfection | | |
| enhancer | | Final |
| supplements | Name | Concentration |
| 1 | Insulin | 10 ug/ml |
| 2 | Holo-Transferrin | 160 ug/ml |
| 3 | Sodium Selenite (ITS liquid) | 5 ng/ml |
| 4 | L-Ascorbic Acid | 50 ug/ml |
| | | (1 mM) |
| 5 | GM-CSF | (10 ng/ml) |
| 6 | SCF | 300 ng/ml |
| 7 | VEGF | 20 ng/ml |
| 8 | IGF-I | 40 ng/ml |
| 9 | IGF-II | 200 ng/ml |
| 10 | IL-3 | 5 ng/ml |
| 11 | Fit3-L | 50 ng/ml |
| 12 | Thrombopoietin (TPO) | 50 ng/ml |
| 13 | Dexamethasone | 1 uM |
| 14 | Fatty Acid free BSA | 1% |
| 15 | 1-Thioglycerol | 0.45 mM |
| 16 | BMP4 | 10 ng/ml |
| 17 | IL-1a | 5 ng/ml |
| 18 | IL-1b | 5 ng/ml |
| 19 | IL-2 | 2 ng/ml |
| 20 | IL-4 | 5 ng/ml |
| 21 | IL-5 | 5 ng/ml |
| 22 | IL-6 | 10 ng/ml |
| 23 | IL-7 | 10 ng/ml |
| 24 | IL-8 | 5 ng/ml |
| 25 | IL-10 | 5 ng/ml |
| 26 | IL-12 | 5 ng/ml |
| 27 | IL-16 | 5 ng/ml |
| 28 | IL-32a | 5 ng/ml |
| 29 | IL-36R2 | 5 ng/ml |

### Example 4—Results of Ad MSCs Isolation, Culture and Conversion to HS/PCs Identification of the Adipose-Derived Mesenchymal Stem Cells (Ad MSC) Isolation with a Modified One-Step Method for Both Human and Mouse Fat Tissues

FIG. 1 shows there is a positive Oil Red O Stained in adipose derived mesenchymal stem cells (Ad MSC) from both human and mice, as Oil Red O Staining is one of the common methods to demonstrate adipocyte.

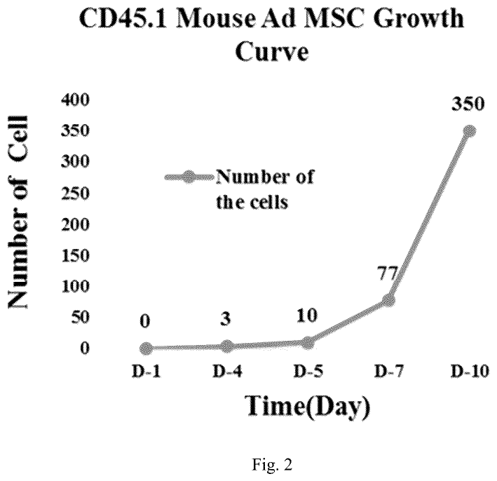

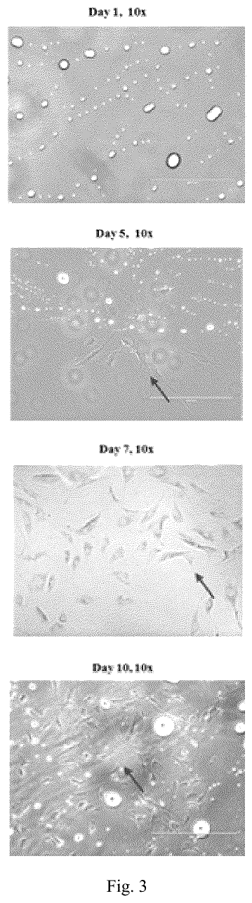

FIGS. 2-3 show that Ad MSCs culture growth curve, that the Ad MSCs growth increased linearly after 7 days of culture.

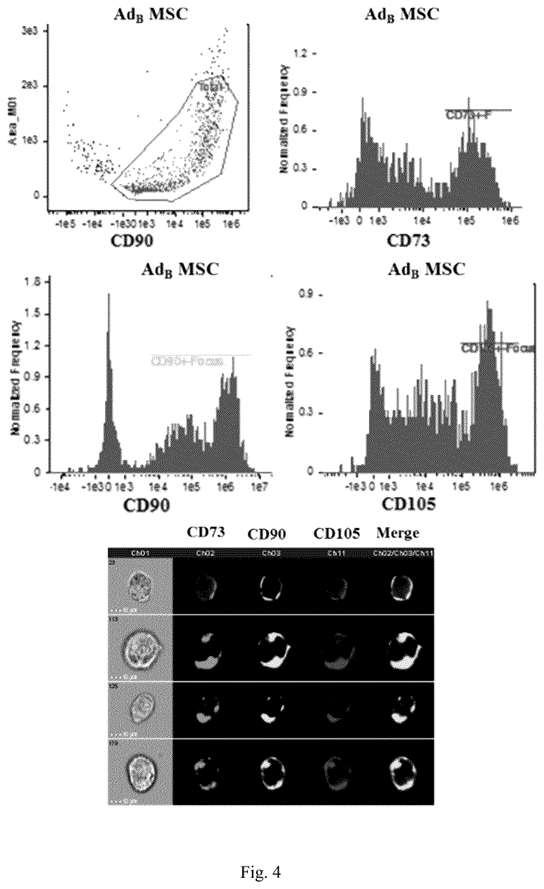

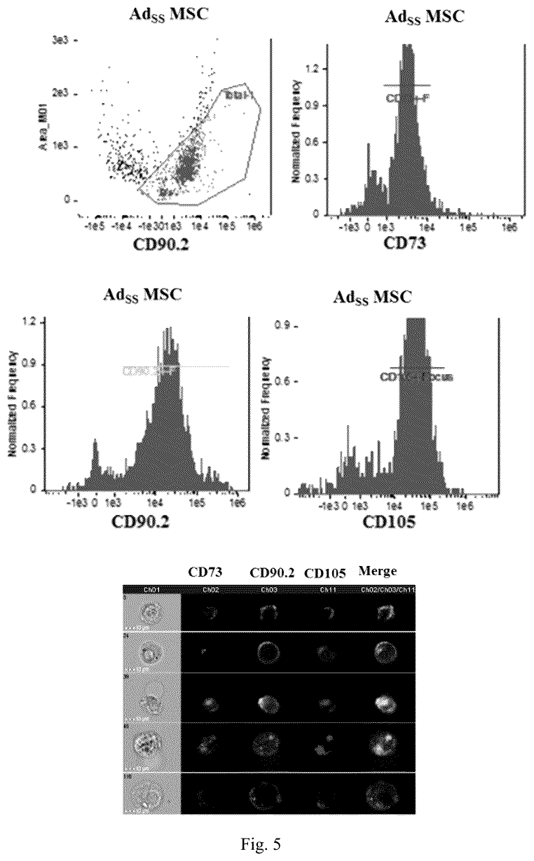

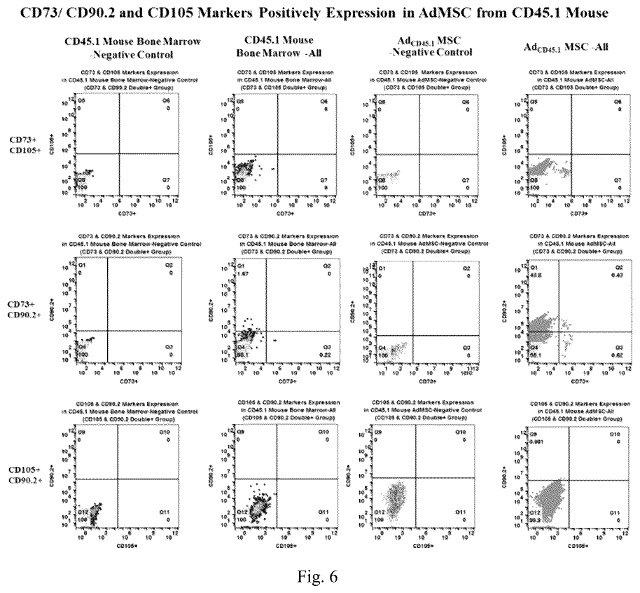

FIGS. 4-6 are Imaging Flow Cytometry results. They show the Ad MSCs-specific markers CD73, CD90, CD90.2 and CD105 are positively expressed in both human and mouse adipose derived mesenchymal stem cells (Ad MSC). However, CD90.2 marker is expressed in mouse bone marrow and CD 73 and CD105 was not expressed in mouse bone marrow. Therefore, the cells are the adipose derived mesenchymal stem cells (Ad MSC) isolated by one-step method isolation approach. The one-step isolation and Ad MSCs culture medium system can be use in this flied.

Conversion of Primary Ad MSCs into Hematopoietic Stem/Progenitor Cells (HS/PC) with UL™-h or m HS/PC-C Medium Vitro.

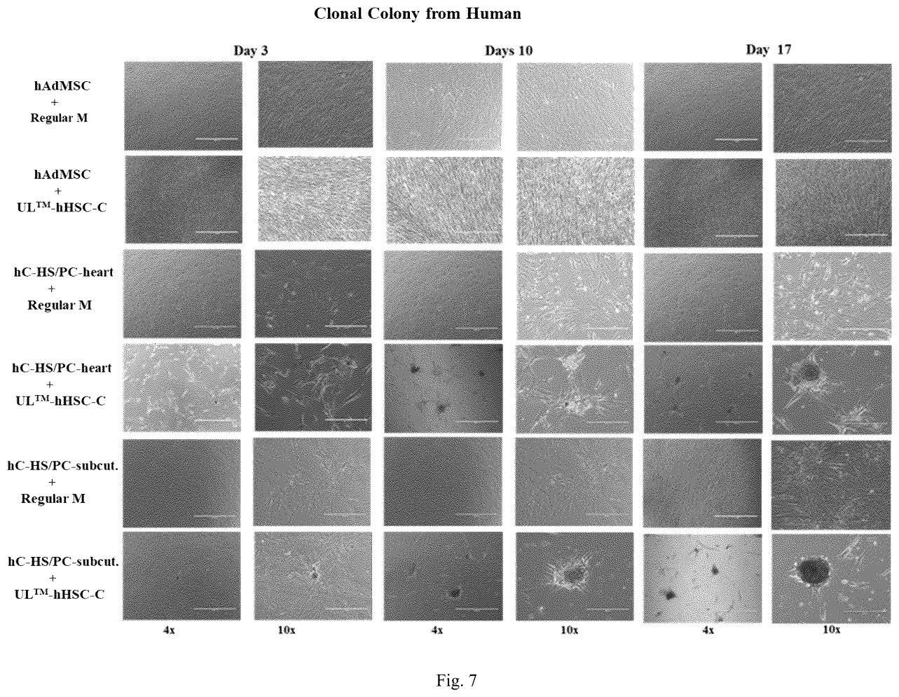

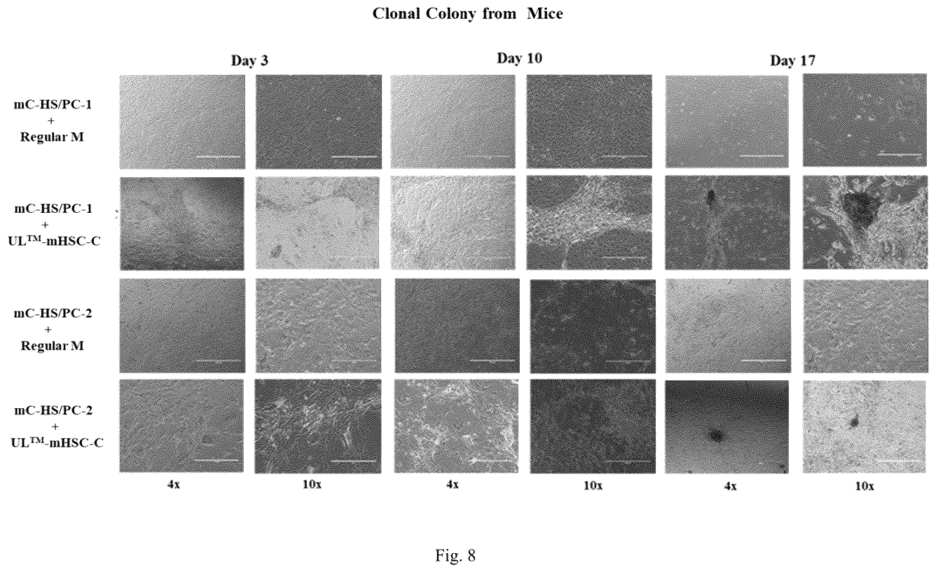

FIGS. 7-8 show converting Ad MSCs to HS/PCs using the UL™-h or m HS/PC-C completely medium for culture. The growth of the converted HS/PCs began to form clones from the third day, and clones became more obvious, and the size of clones increases with the extension of culture time. This result is almost same for both human and mouse cells. There are no clones in the Ad MSCs with the UL™-HS/PC-C completely medium.

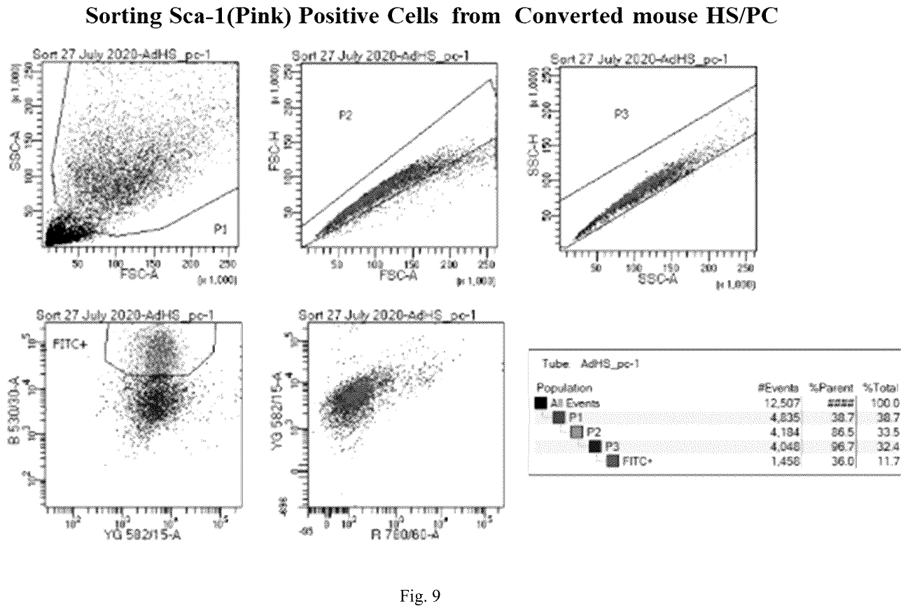

FIG. 9 shows the converted mouse HS/PCs that were stained with FITC-Sca-1 (pink) marker (Sca-1 is hematopoietic stem cell specific marker for mice) and sorting by Flow cytometry. The FITC-Sca-1 positive cell is 11.7% in total sorted cells. It means the HS/PCs converted rate is 11.7%, which is significantly higher than any existing technologies.

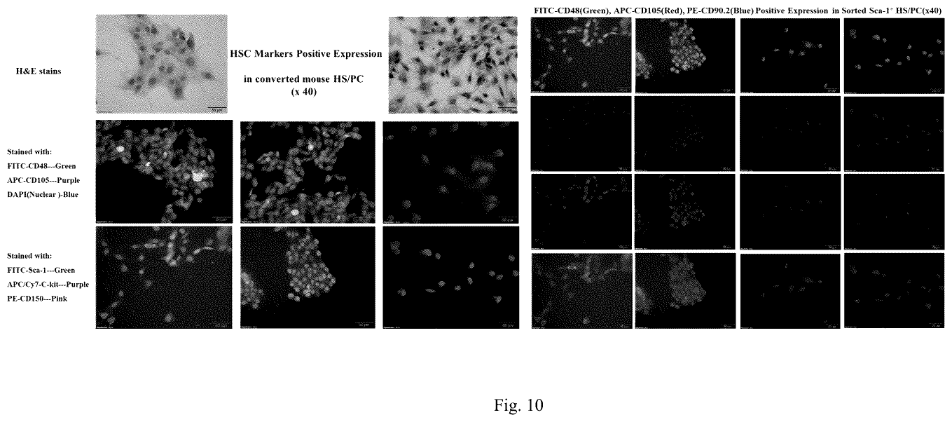

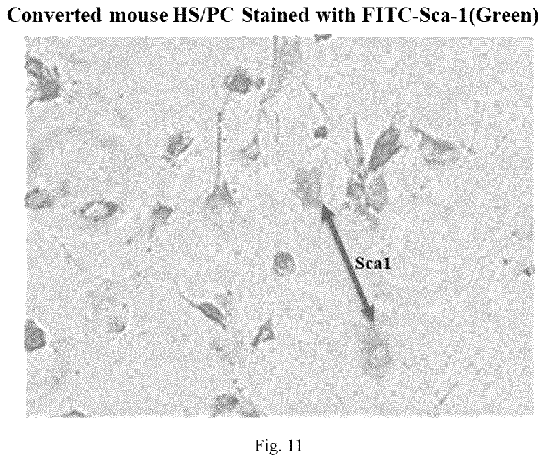

FIG. 10-11 show that CD90.2 (blue) c-kit (CD117) (purple), CD48 (green), CD105 (red), CD150 (pink) markers (mouse hematopoietic stem cells specific marker) are positively expressed in converted Sca-1 Positive mouse HS/PC(D). The FITC-Sca-1 marker (green) shows positive expression in live cultured converted mouse HS/PCs(E), as shown in FIG. 11.

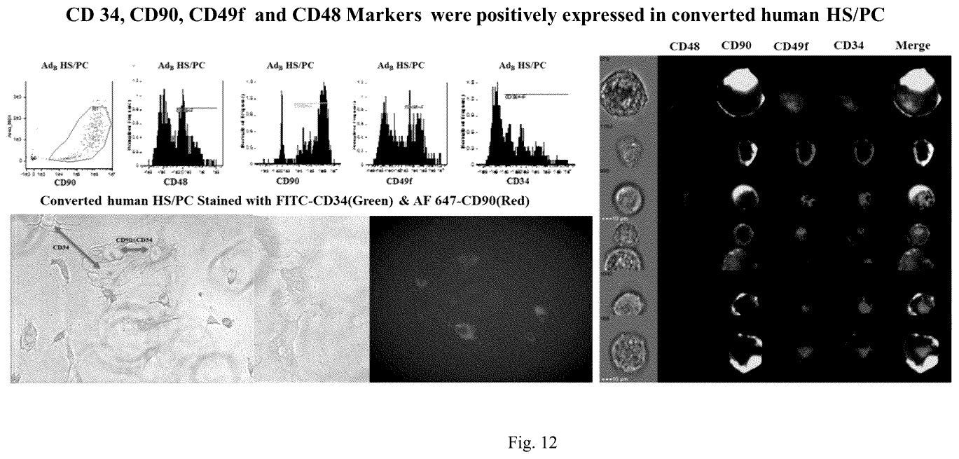

FIG. 12 shows flow cytometry results reflecting that CD34 (green, red), CD90 (yellow, red) and CD49f (pink) markers are positive expressions in converted HS/PC from human cells.

Therefore, as it can be observed in FIGS. 7-12, both human and mouse Ad MSCs can form clones and have positive expression of hematopoietic stem cell-specific markers under UL™-HS/PC-C medium without introduced any exogenous genes in vitro. However, there is no clone formation in the Ad MSCs cultured in the UL™-HS/PC-C medium.

Differentiation System for Converted HS/PCs into Various Lineages of Peripheral Blood Cells Both In Vitro and In Vivo
Converted HS/PCs Differentiate into Glycophorin a (CD235a for Human and TER 119 for Mouse) and Fetal Hemoglobin Marker Expression:

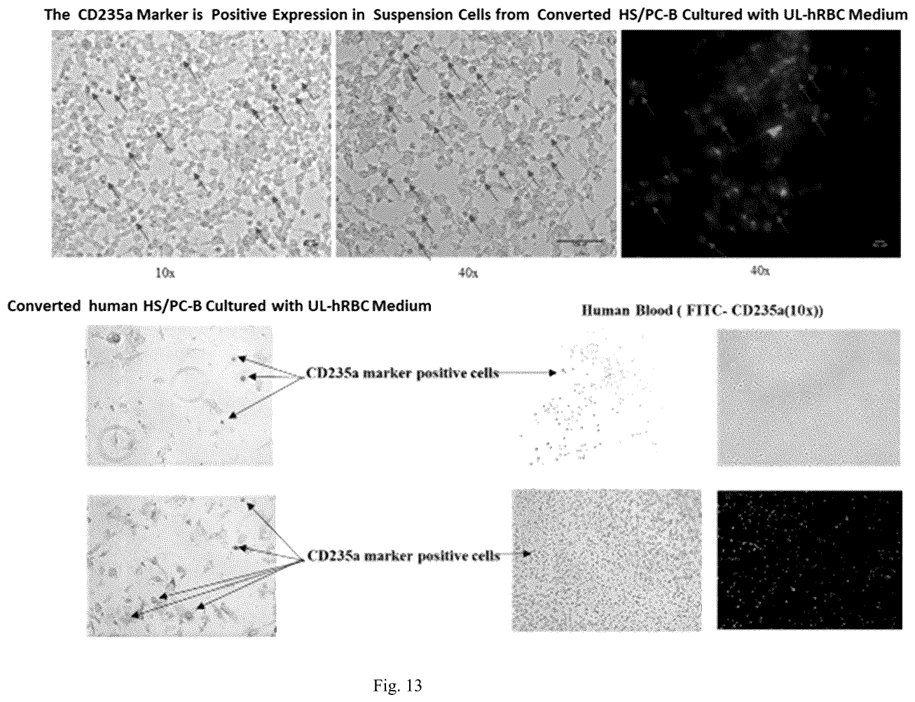

In one embodiment, FIGS. 13-19 shows in vitro and in vivo results.

FIG. 13 shows that Glycophorin A(CD235a) (green) marker was positively expressed in supernatant cells from human converted HS/PC under UL™-hRBC medium after 18 to 23 days culture in FIG. 13 top panel. FIG. 13 bottom panel shows the control from human blood RBC stained with CD235a (green).

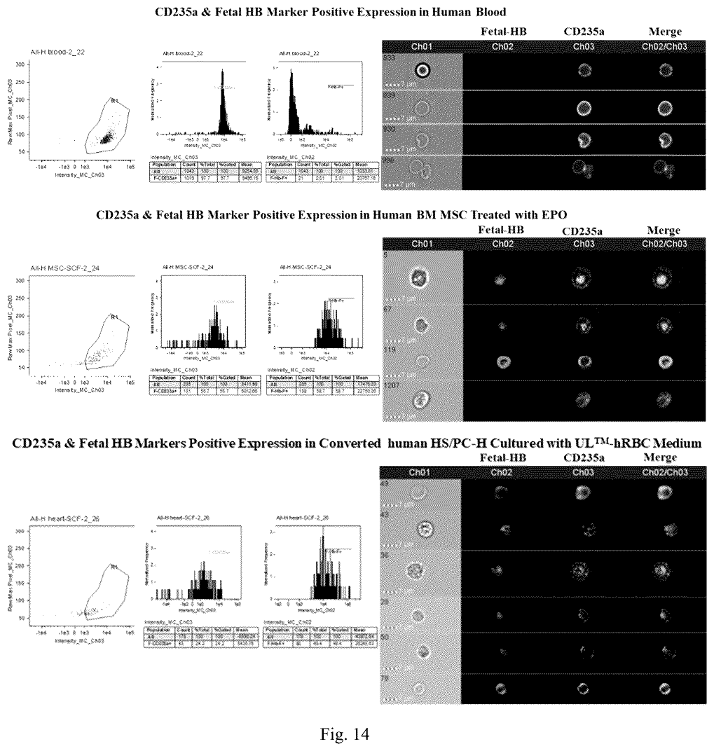

FIG. 14 shows Image Flow Cytometry results demonstrating that converted human HS/PCs cultured in UL™-hRBC completely medium after 18 to 23 days, there are new suspended cells in the culture medium and positive expression of specific Glycophorin A(CD235a) (yellow) and Fetal-Hb (green) markers in FIG. 14 bottom panel. Figure B top panel is the positive control from human blood RBC stained with CD235a and Fetal-Hb. There is only CD235a (yellow) marker expression in the human blood RBC and no Fetal-Hb (green) marker expression in the human RBC, because Fetal-Hb is the main oxygen carrier protein in the fetus.

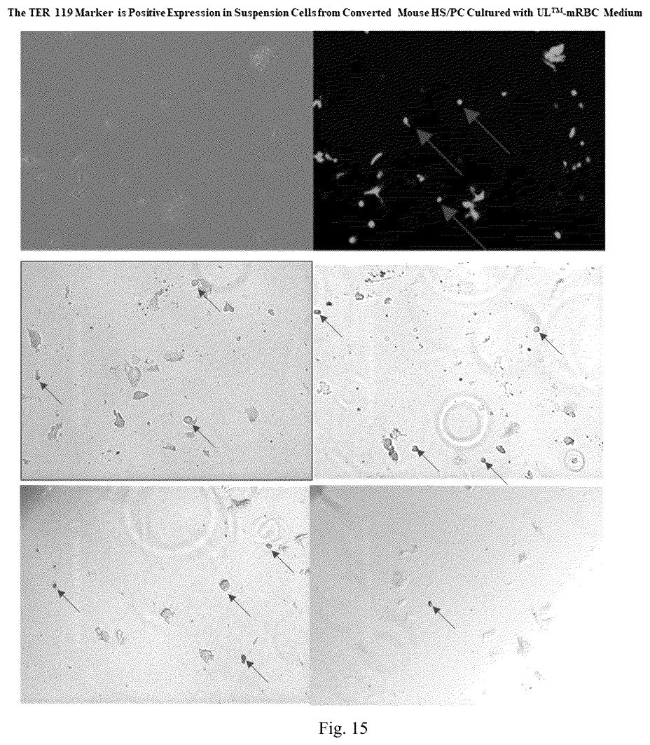

FIG. 15 shows Glycophorin A (TER 119) (green) marker was positively expressed in supernatant cells from mouse converted HS/PC under UL™-mRBC Medium after 18 to 23 days culture. TER 119 is a special marker for mouse red blood cells.

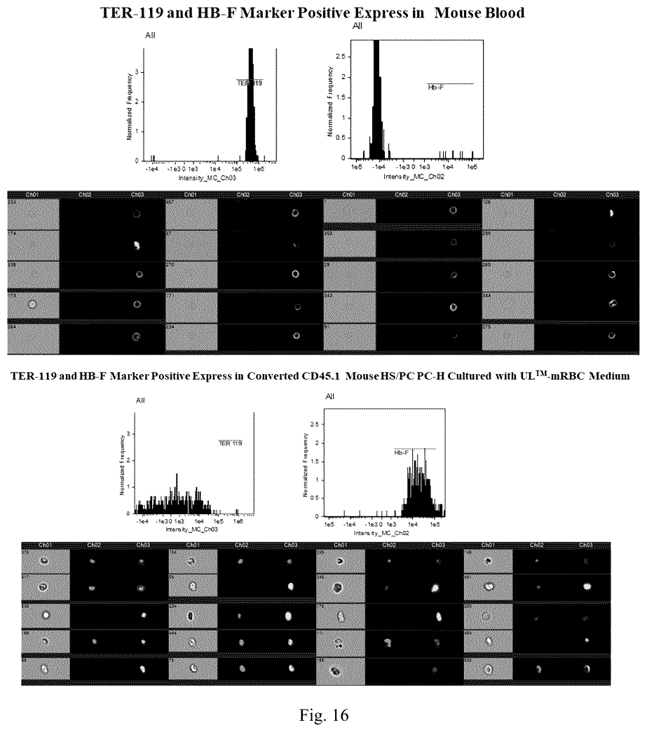

FIG. 16 shows Image Flow Cytometry results demonstrating that converted mouse HS/PCs cultured in UL™-mRBC completely medium after 18 to 23 days, there are new suspended cells in the culture medium and suspended cells are positive expression of specific TER 119 (yellow) and Fetal-Hb (green) markers on FIG. 16 bottom panel. FIG. 16 top panels are the positive control from mouse blood RBC stained with TER 119 (yellow) and Fetal-Hb (green). There is no Fetal-Hb (green) marker expression in the mouse blood RBC, because Fetal-Hb is the main oxygen carrier protein in the fetus.

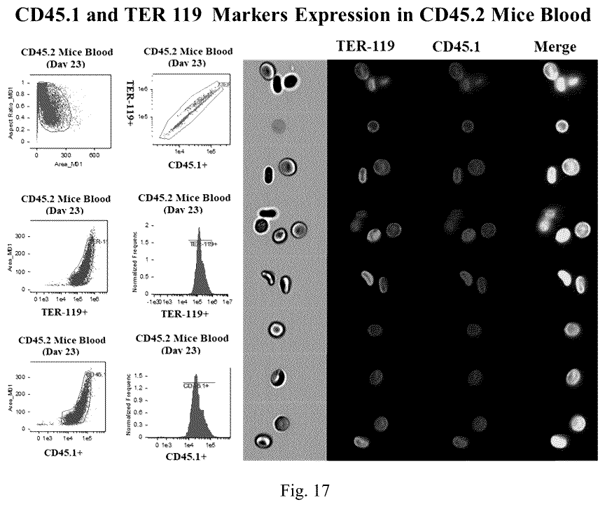

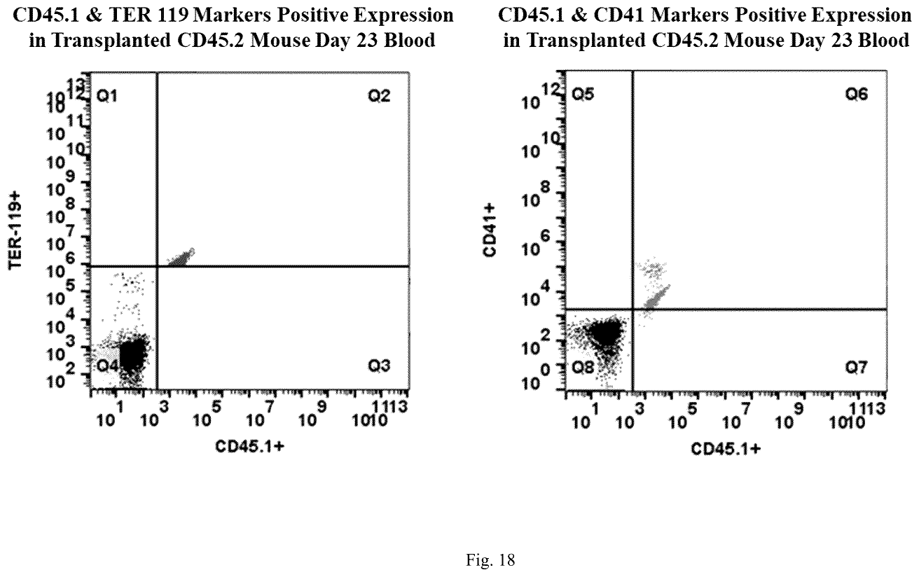

FIGS. 17-18 show Image Flow Cytometry results demonstrating that there is CD45.1 (red), TER 119 (green) and CD41 markers positive expression in CD45.1 mouse converted HS/PCs transplanted CD45.2 mouse blood cells on day 23. CD41 is a special marker for mouse platelets.

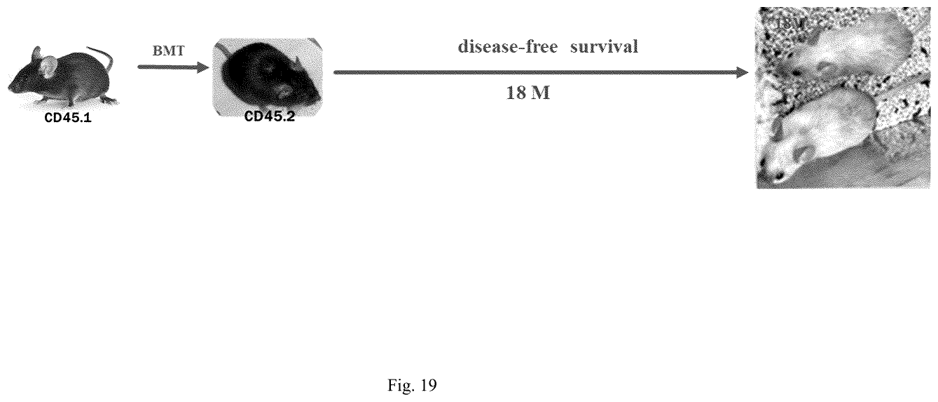

FIG. 19 shows transplantation of the converted HS/PCs of CD45.1 mice into 6 of the CD45.2 mice, the 4 of the CD45.2 mice were healthy survived for 18 months.

Therefore, it can be concluded that (1) Glycophorin A(CD235a) (TER 119), and Fetal-Hb markers were positively expressed in supernatant cells from human and mouse converted HS/PC culture under UL™-h or m RBC Medium after 18 to 23 days culture in vitro; (2) Glycophorin A (TER 119), CD41 and CD45.1 markers were positively expressed in CD45.1 mouse converted HS/PCs transplanted CD45.2 mouse blood cells CD45.2 mouse blood on 23 days; (3) the converted hematopoietic stem cells (HS/PCs) differentiate into cells with Fetal-Hb, Glycophorin A and CD41 markers of positive expression under UL™-h or m RBC Medium in vitro and in vivo; (4) transplantation results demonstrated that converted HS/PCs can rebuild the immune system in vivo and are safe for reinfusing into the body.

This further confirms that HS/PCs converted from AdMSCs can differentiate into Fetal-Hb, Glycophorin A and CD41 markers of positive expression blood cells and under innovation UL™-h or m RBC system without inserting any exogenous genes in vitro and in vivo.

Differentiation Converted HS/PCs into Leukocytes Marker Positive Cells In Vitro and In Vivo by UL™-Human or Mouse White Blood Cell Kit (UL™-h or m WBC Kit).

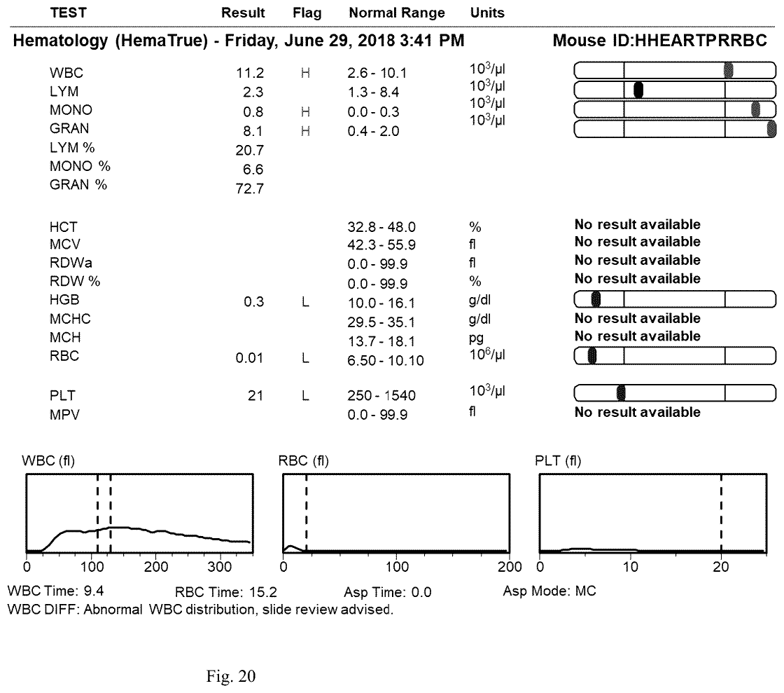

In one embodiment, FIGS. 20-22 show in vitro and in vivo results.

FIG. 20 shows CBC results reflecting the cells in supernatant from mouse converted HS/PCs under UL™-mWBC medium at 25 days culture. The WBC are 11.2×10³/ul, LYM (lymphocytes) are 2.3×10³/ul, MONO (monocytes) are 0.8×10³/ul, and GRAN (granulocytes) are 8.1×10³/ul.

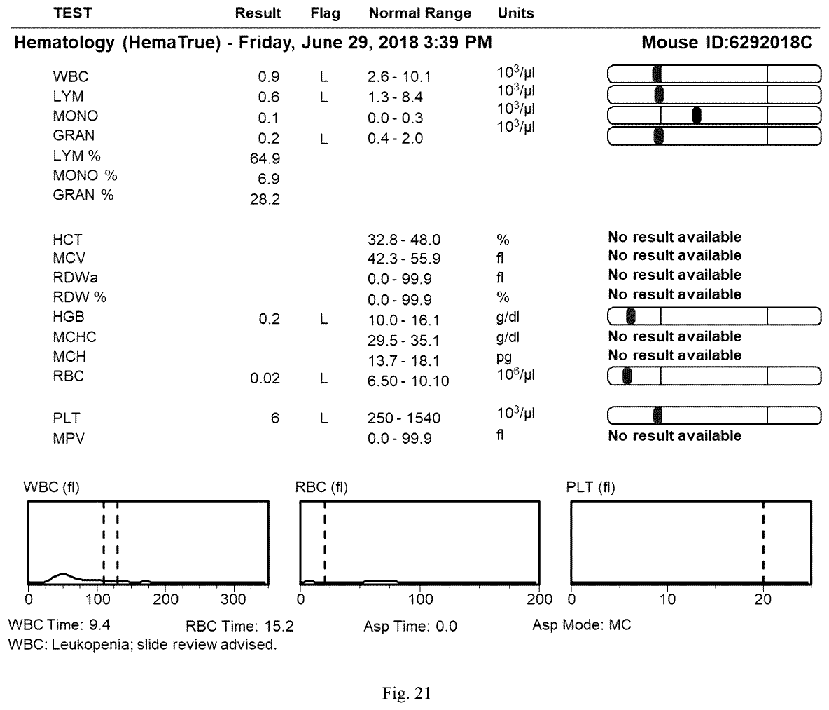

FIG. 21 reflects CBC results showing that the cells in supernatant from mouse converted HS/PCs under regular medium at day 25 days culture. The WBCs are 0.9×10³/ul, LYM (lymphocytes) are 0.6×10³/ul, MONO (monocytes) is 0.1×10³/ul, and GRAN (granulocytes) are 0.2×10³/ul. (control)

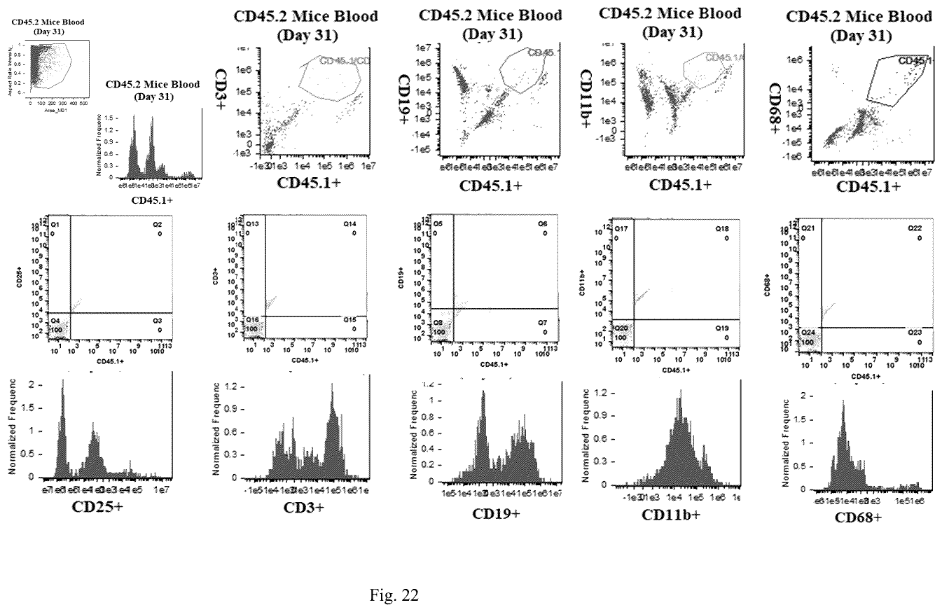

FIG. 22 is flow cytometry analysis showing that there is positive expression of the CD45.1 mice of leukocyte markers (CD45.1, CD3/CD45.1, CD19/CD45.1, CD25/CD45.1, CD11b/CD45.1 and CD68/CD45.1) in the CD45.2 mouse peripheral blood which was transplanted CD45.1 mouse converted HS/PCs at 31st day.

Therefore, this further confirms that HS/PCs converted from Ad MSCs can differentiate into blood cells with positive expression multi-leukocyte markers under our innovation UL™-h or m WBC medium without inserting any exogenous genes in vitro and in vivo.

### Enhancing System for Gene Delivery of the Converted HS/PCs In Vitro (UL™-TFK).

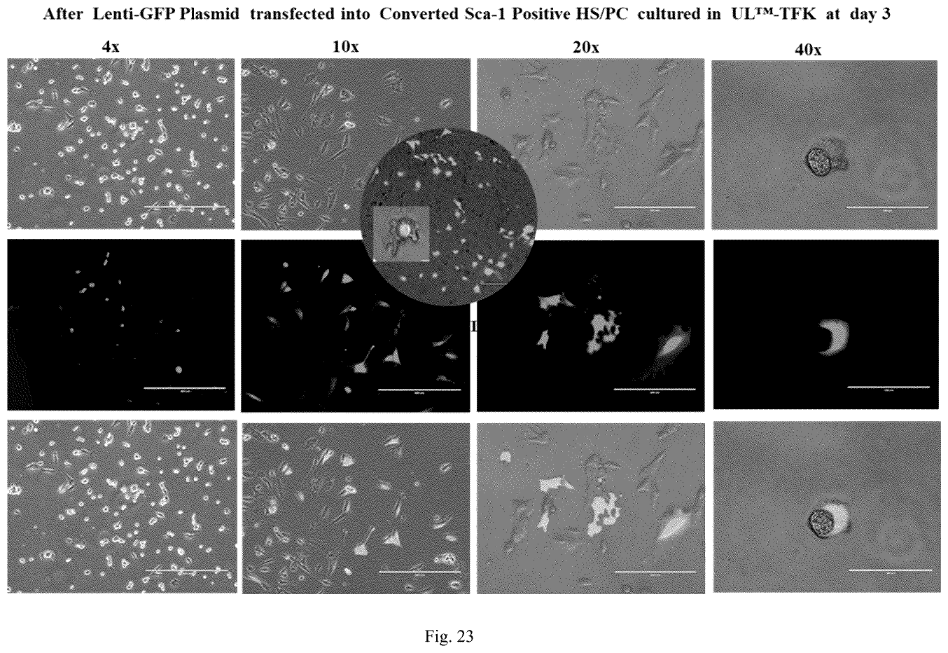

FIG. 23 shows there are at least 25 GFP positive cells (green) in the converted HS/PCs transfected with pLenti-GFP in culture under UL™-TFK medium condition on day 3.

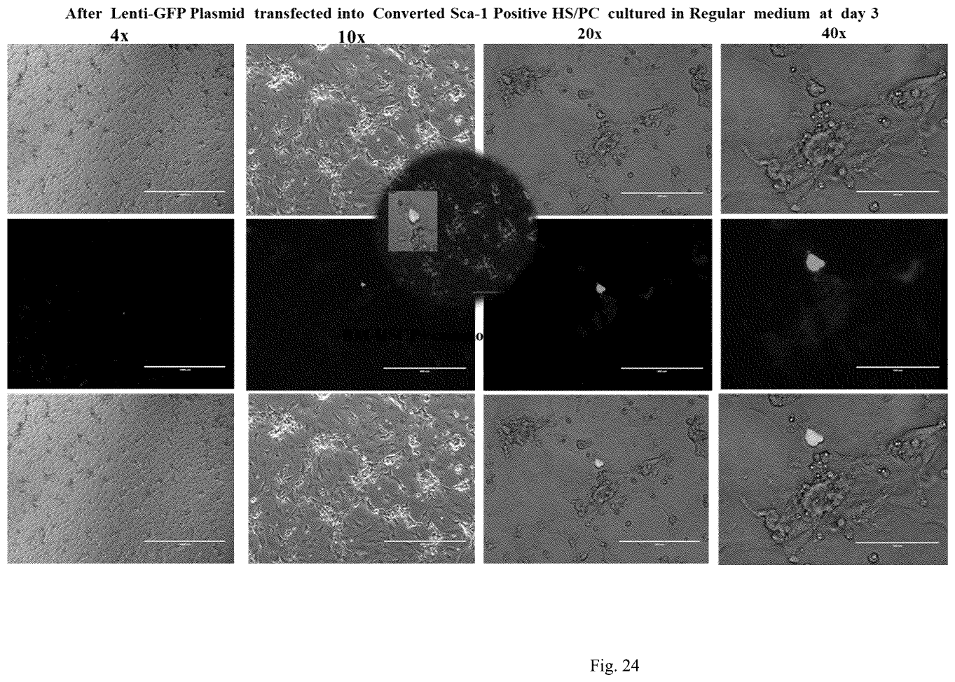

FIG. 24 shows only 1 GFP positive cell shown in the converted HS/PCs transfected with pLenti-GFP culture in the regular medium condition on day 3.

Comparing the gene delivery efficiencies of UL™-TFK medium and regular medium, the transfection efficiency of UL™-TFK medium is about 25-fold higher than the regular medium.

Therefore, the converted HS/PCs derived from Ad MSCs can be good gene delivery carry-on cells. Also the converted HS/PCs can differentiates into desired cells in a targeted manner (like T cell, B cell, NK cell and etc.)

Moreover, the UL™-TFK can increase gene transfection efficiency. That combined converted HS/PCs and UL™-TFK together addresses some of the key hurdles experienced in gene therapy today, specifically the challenges of culturing gene-carrying cells and transfection in vitro setting.

### Example 5—Protocols for Ad MSCs Isolation, Expansion, and Conversion into HS/PCs; and Differentiation Converted HS/PCs into Various Blood Cells

### Methods and Procedures

The fat tissues from 5 different strains total 10 mice and 3 human Ad MSCs cell lines.

In one embodiment, the present invention discloses an in vitro method.

### Ad MSCs Isolation and Expansion Culture:

In one embodiment, the present invention discloses an automatic process for Ad MSCs isolation and expansion culture.
1. 0.5 g mouse intra-abdominal fat tissues wash with wash buffer for 3 times, cut into 0.5 mm² small pieces with scissors under sterile hood. Add 2 ml wash buffer to wash dish and transfer everything into grinder.
2. Selected program at the UL-Automatic multifunctional single cell processor and Press start button.
3. Transfer the cell flask from the automatic multifunctional single cell processor into the cell culture incubator at 37° C. with 5% CO₂ in humid conditions. Change the medium twice a week. One cannot see any cells in the dish for the first two or three days.

In one embodiment, the present invention discloses a manual process for Ad MSCs isolation and expansion culture.
1. 0.5 g mouse intra-abdominal fat tissues washed with wash buffer for 3 times, cut into 0.1-3 mm² small pieces with scissors under sterile hood. Add 2 ml wash buffer to wash dish and transfer everything into a 15 cm sterile centrifuge tube with 8 ml enzyme digestive reagent (UL™-TMSCs-IS kit). Incubate at 37° C. for 45-60 minutes with shake.
2. When the liquid turns into opaque (showing that the fat tissue is completely decomposed), transfer all liquid into a new 50 ml sterile centrifuge tube equipped with a 70 mm cell strainer, then pour the liquid into a 100 mm cell strainer in 100 mm culture dish and rinse the filter membrane with 10 ml washing solution. Tilt the culture dish, use a glass pipette to suck the liquid from the bottom of the culture dish by vacuum suction (try to make the uppermost liquid surface contact the bottom of the culture dish). Incubate the dish at room temperature for 10 minutes, then gently add wash solution. Aspirate the lotion as before and repeat 2 times.
3. Adding 10 ml of UL™-Ad MSC Expansion Medium (UL™-Ad MSC-E) into the dish, culture the cell dish in incubator at 37° C. with 5% CO₂ in humidly condition. Change the medium twice a week. One cannot see any cells in the dish for the first two or three days.

### Converting Ad MSCs to HS/PCs In Vitro:

1. When the Ad MSCs grow around 90% confluence, wash cells with 1×PBS.
2. Add 5 ml to the 60 mm plate or 8 ml to 25 cm Flask UL™-HS/PC-C completely medium for 48 hours.
3. Change UL™-HS/PC-C completely medium to UL™-HS/PC-C medium-2 to continue culture. Change medium twice per week.

One usually can convert HS/PCs around 6 to 8 days. Indentation of the cells by Flow Cytometry analysis.
4. When the clones grow up, subculture according to the manual.
   Differentiation Converted into RBC & Platelets Markers Cells In Vitro.
1. When the converted HS/PCs grow around 90% confluence, wash cells with 1×PBS.
2. Add 5 ml to the 60 mm plate or 8 ml to 25 cm Flask UL™-RBC completely medium to culture the cells in incubator at 37° C. with 5% CO₂ in humid conditions.

The medium should be added twice a week for the first 10 to 15 days.
3. Just add 5 ml to the 60 mm plate or 8 ml to 25 cm Flask UL™-RBC completely medium to culture the cells in incubator at 37° C. with 5% CO₂ in humid conditions.
4. Just add 10 ml or 20 ml UL™-RBC completely medium twice a week from day 11 to 23. Do not discard the old medium please and carefully observe the cell growth. If you see new small dark cells in the culture medium, please collect them for further research. At the same time, add fresh medium to the dish for further culture.
   Differentiation Converted into WBC Markers Cells In Vitro.
1. When the converted HS/PCs grow around 90% confluence, wash cells with 1×PBS.
2. Add 5 ml to the 60 mm plate or 8 ml to 25 cm Flask UL™-WBC completely medium to culture the cells in incubator at 37° C. with 5% CO₂ in humid conditions.

The medium should be added twice a week for the first 15 to 38 days.
3. Just add 5 ml to the 60 mm plate or 8 ml to 25 cm Flask UL™-WBC completely medium to culture the cells in incubator at 37° C. with 5% CO₂ in humid conditions.
4. Just add 10 ml or 20 ml UL™-RBC completely medium twice a week from day 16. Do not discard the old medium please and carefully observe the cell growth. If new small dark cells appear in the culture medium, collect them for further research. At the same time, add fresh medium to the dish for further culture.

### Enhancing System for Gene Delivery of the Converted HS/PCs In Vitro.

1. Isolation and culture the Ad MSCs with UL™ Ad MSCs-IS kit and UL™-Ad MSC-E kit.
2. Converted Ad MSCs to HS/PCs with UL™-HS/PC-C and culture cells for 6 to 8 days, then subculture the converted HS/PCs at 70% or 80% confluence.
3. Set transfection experiment by any transfection method.
4. After overnight transfection experiment, replace the 5 ml or 10 ml UL™-TFK completely medium into the cells. Continue culture at 37° C. with 5% CO₂ in humid conditions.
5. The target cells can be selected with any method and sorting the positive cells for future purpose. Also transfected of converted HS/PCs can differentiates into desired cells in a targeted manner (like T cell, B cell, NK cell on so on) too. At this point, one can avoid the cell lack that loses the treatment chance from patient.
6. UL™-Ad MSC-E kit. Can give more chance for cells expansion and subculture. The details, please follow the manual.

### In Vitro Procedures:

1. Isolation and culture Ad MSCs from CD45.1 mice are disclosed above.
2. Converting Ad MSCs to HS/PCs for CD45.1 mice are disclosed below.
3. After sorting the positive converted HS/PCs, culture the cells with UL™-Ad MSC-E medium.
4. When grown enough positive markers HS/PCs, collecting the cell, and washing with 1×D-PBC for 3 to 4 times to completely wash away protein components.
5. At the transplantation the same day, isolation CD45.2 mice bone marrow cells.
6. Suspension of the converted CD45.1-HS/PCs in 0.1 ml saline with 1-2×10⁷ cells and suspension the CD45.2 BM cell in 0.1 ml saline with 1-2×10⁷ cells. Then mix both cells well and keep the cells on ice before injecting them into the mice.
7. The CD45.2 recipient mice require radiation exposure twice; the first radiation is 4 hours early and second radiation needs done at 1 hour early before the injecting the cells into to the CD45.2 recipient.
8. Injecting the mixed cell into the recipient mouse through the facial muscles.
9. Keep CD45.2 recipient mice in healthy condition and watch carefully.
10. Start checking the effect of transplantation on mice for about three weeks by collecting eye blood or tail blood.
11. Analysis by Flow Cytometry.

### Analysis:

1. Oil Red O Stained: practiced accordingly to common knowledge in the field.
2. Flow Cytometry analysis: Detail for cell labeling according to the manufacture manual.
   A. Ad MSCs panel:
For Human: FITC-CD90, PE-CD73, APC-CD105
For Mice: FITC-CD90.2, PE-CD73, APC-105

### B. HSCs Panel:

For Human:
Brilliant Violet 421™ anti-human/mouse CD49f
Brilliant Violet 605™ anti-mouse CD45 Antibody
Alexa Fluor® 647 anti-human CD34 Antibody
FITC anti-human CD48 Antibody
PE/Cy5 anti-human CD135 (Flt-3/Flk-2)
PE anti-human CD90 (Thy1) Antibody
APC/Cy7 anti-human CD10 Antibody
For Mice:
Brilliant Violet 421™ anti-human/mouse CD49f
Brilliant Violet 711™ anti-mouse Ly-6A/E (Sea-1) Antibody
PE anti-mouse CD90.2 Antibody
PE/Cy7 anti-mouse CD150 (SLAM) Antibody
PE/Cy5 anti-mouse CD135 Antibody
Brilliant Violet 605™ anti-mouse CD45 Antibody
Alexa Fluor® 647 anti-mouse CD34 Antibody
FITC anti-mouse CD48 Antibody
APC/Cy7 anti-mouse CD117 (c-kit) Antibody

### C. RBC Panel:

For Human:
FITC anti-human CD235a
PE anti-human CD62P (P-Selectin) Antibody
APC anti-human CD42b Antibody
For Mice:
FITC anti-mouse TER-119/Erythroid Cells
PE anti-mouse/rat CD62P (P-selectin) Antibody
APC anti-mouse CD41 Antibody

### D. WBC Panel:

For Human:
Brilliant Violet 421™ anti-human CD25 Antibody
Brilliant Violet 510™ anti-human CD19 Antibody
Brilliant Violet 711™ anti-human CD14 Antibody
Anti-human CD3 PE-Cy5/CD4 PE/CD8 FITC Cocktail
PE/Cy7 anti-human CD1c Antibody
APC anti-human CD56 (NCAM) Antibody
APC/Cy7 anti-human CD68 Antibody
For Mice:
Brilliant Violet 421™ anti-mouse CD25 Antibody
Brilliant Violet 510™ anti-mouse CD19 Antibody
Brilliant Violet 711™ anti-mouse CD115 (CSF-1R)
PE anti-mouse CD3 Antibody
PE/Cy7 anti-mouse CD11c Antibody
APC anti-mouse CD49b (pan-NK cells) Antibody
APC/Cy7 anti-mouse CD68 Antibody

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of producing hematopoietic stem/progenitor cells (HS/PCs) and blood cells from adipose derived mesenchymal stem cells (Ad MSCs), the method comprising:
obtaining biological cells of a subject;
expanding the biological cells;
obtaining a pure Ad MSCs cell line from the expanded biological cells;
converting the pure Ad MSCs cell line into HS/PCs in vitro;
expanding the converted HS/PCs in vitro; and
obtaining the expanded HS/PCs.

2. The method according to claim 1, the methods further comprising:
differentiating the expanded HS/PCs into a blood cell line;
wherein the blood cell line comprises at least one of white blood cells (WBC), red blood cells (RBC), and platelets.

3. The method according to claim 1, wherein the step of converting the pure Ad MSCs cell line into the HS/PCs does not use any exogenous genes.

4. The method according to claim 1, wherein the step of obtaining the pure Ad MSCs cell line comprises:
isolating the pure Ad MSCs cell line from the biological sample; and
culturing the pure Ad MSCs cell line.

5. The method according to claim 1, wherein the step of converting the pure Ad MSCs cell line into the HS/PCs comprises:
using a HS/PCs converting kit.

6. The method according to claim 5, wherein the HS/PCs converting kit comprises:
a basic Ad MSCs culture medium;
a basic nutrition supplement; and
a basic converting supplement mixture.

7. The method according to claim 6, wherein the step of converting the pure Ad MSCs cell line into the HS/PCs comprises:
preparing a HS/PC-C completely medium by mixing the basic nutrition supplement and the basic converting supplement mixture into the basic Ad MSCs culture medium;
adding the HS/PC-C completely medium to the pure Ad MSCs cell line obtained; and
incubating the pure Ad MSCs cell line in the HS/PC-C completely medium for a period of time.

8. The method according to claim 5, wherein the step of converting the pure Ad MSCs cell line into the HS/PCs has a conversion efficiency of more than 10%.

9. The method according to claim 5, wherein the converted HS/PCs have a gene transfection efficiency 25-fold higher than that of regular converted HS/PCs which are not converted by the HS/PCs converting kit.

10. The method according to claim 5, wherein the converted HS/PCs comprises gene carry-on cells.

11. A HS/PCs converting kit for converting adipose derived mesenchymal stem cells (Ad MSCs) into hematopoietic stem/progenitor cells (HS/PCs), the kit comprising:
a basic Ad MSCs culture medium;
a basic nutrition supplement; and
a basic converting supplement mixture,
wherein the kit converts the Ad MSCs into HS/PCs.

12. The kit according to claim 11, wherein the basic nutrition supplement comprises fetal bovine serum and horse serum.

13. The kit according to claim 11, wherein the basic converting supplement mixture comprises at least one of insulin, holo-transferrin, sodium selenite (ITS liquid), L-ascorbic acid, GM-CSF, SCF, VEGF, IGF-I, IGF-II, IL-3, Flt3-L, thrombopoietin (TPO), dexamethasone, fatty acid free BSA, 1-thioglycerol, SB431542, CHIR99021, and Y-27632.

14. The kit according to claim 14, wherein the basic converting supplement mixture comprises insulin, holo-transferrin, sodium selenite (ITS liquid), L-ascorbic acid, GM-CSF, SCF, VEGF, IGF-I, IGF-II, IL-3, Flt3-L, thrombopoietin (TPO), dexamethasone, fatty acid free BSA, 1-thioglycerol, SB431542, CHIR99021, and Y-27632.

15. A red blood cells (RBCs) and platelets differentiation kit for differentiating hematopoietic stem/progenitor cells (HS/PCs) into RBCs and platelets, the kit comprising:
a basic cell culture medium;
a basic nutrition supplement; and
a RBCs supplement mixture;
wherein the kit differentiates the HS/PCs into at least one of RBCs and platelets.

16. The kit according to claim 15, wherein the basic nutrition supplement comprises fetal bovine serum and horse serum.

17. The kit according to claim 15, wherein the RBCs supplement mixture comprises at least one of insulin, holo-transferrin, sodium selenite (ITS liquid), L-ascorbic acid, SCF, VEGF, IGF-I, IGF-II, IL-3, Flt3-L, thrombopoietin (TPO), dexamethasone, fatty acid free BSA, 1-thioglycerol, EPO, ferrous sulfate, PDGF BB, activin A.

18. A white blood cells (WBCs) differentiation kit for differentiating hematopoietic stem/progenitor cells (HS/PCs) into WBCs, the kit comprising:
a basic cell culture medium;
a basic nutrition supplement; and
a WBCs supplement mixture;
wherein the kit differentiates the HS/PCs into WBCs.

19. The kit according to claim 18, wherein the basic nutrition supplement comprises fetal bovine serum and horse serum.

20. The kit according to claim 18, wherein the basic converting supplement mixture comprises at least one of insulin, holo-transferrin, sodium selenite (ITS liquid), L-ascorbic acid, SCF, VEGF, IGF-I, IGF-II, IL-3, Flt3-L, thrombopoietin (TPO), dexamethasone, fatty acid free BSA, 1-thioglycerol, BMP4, IL-7, IL-12, IL-11, IL-6, IL-2, FGF-b, TGF-a, TGF-b1, TGF-b2, TGF-b3, IL-1a, IL-1b, IL-4, IL-5, IL-8, IL-10, IL-12, IL-32a, mIL-36R2, GM-CSF.

21. An automatic multifunctional single cell processor and culture device, the device comprising:
a housing;
at least one small fluid volume dispenser system disposed inside the housing; and
a tissue grinding system disposed below the small fluid volume dispenser system and in the housing;
wherein the small fluid volume dispenser system is configured to dispense at least one fluid; and wherein the tissue grinding system receives the at least one fluid.

22. The device according to claim 21, wherein the small fluid volume dispenser system is configured to dispense the at least one fluid in a volume between about 5-10 ml.

23. The device according to claim 21, wherein the small fluid volume dispenser system comprises a cylinder houses one or more pre-filled cartridges.

24. The device according to claim 23, wherein the small fluid volume dispenser system comprises a top needle disposed above the one or more pre-filled cartridges and a bottom needle disposed below the one or more pre-filled cartridges.

25. The device according to claim 24, wherein the top needle and the bottom needle are configured to move in a vertical direction for piercing the pre-filled cartridges.

26. The device according to claim 21, wherein the tissue grinding system comprises a vial mechanically connected to a motor.

27. The device according to claim 26, wherein the vial houses a top grinder plate, a bottom grinder plate, a filter disposed below the top grinder plate and the bottom grinder plate.

28. The device according to claim 26, wherein the top grinder plate and the bottom grinder plate are arranged to form a sample disposing place for receiving a biological sample.

29. The device according to claim 28, wherein the tissue grinding system further comprises a tubing fluidly connected to a bottom of the vial on a first end.

30. The device according to claim 29, wherein the tubing fluidly connected to a dispenser funnel on a second end.

31. The device according to claim 21, wherein the device comprises a cellular flask rotator, wherein the cellular flask rotator comprises multiple magnetic nanoparticles.
